(19) United States
(12) Patent Application Publication
(10) Pub. No.: US 2013/0210005 A1
(43) Pub. Date: Aug. 15, 2013

(54) METHOD FOR DETECTING MYCOBACTERIUM TUBERCULOSIS AND NONTUBERCULOUS MYCOBACTERIA BY USING DUAL REAL-TIME POLYMERASE CHAIN REACTION

(71) Applicant: Jeong-Uk Kim, Gangneung-si (KR)

(72) Inventor: Jeong-Uk Kim, Gangneung-si (KR)

(73) Assignee: UNIVERSITY OF ULSAN FOUNDATION FOR INDUSTRY COOPERATION, Ulsan (KR)

(21) Appl. No.: 13/700,157

(22) PCT Filed: May 27, 2011
(86) PCT No.: PCT/KR2011/003903

§ 371 Date: Apr. 24, 2013

(30) Foreign Application Priority Data
May 27, 2010 (KR) 10-2010-0049692
Jun. 30, 2010 (KR) 10-2010-0062847
Jan. 31, 2011 (KR) 10-2011-0009530

(51) Int. Cl. C12Q 1/68

(52) U.S. Cl. CPC C12Q 1/689
USPC 435/6.11; 536/24.32; 435/6.12

(57) ABSTRACT

Disclosed are a primer set and/or a probe capable of detecting specific nucleotide sequences of MTC and NTM, a kit for the detection of MTC and NTM, comprising the same, and a method for detecting MTC and NTM by duplex real-time PCR using the same. Useful in detecting genes characteristic of MTC and NTM, the primer sets and/or probes, detection kits, and detection methods can be applied as the clinical diagnosis of diseases caused by MTC and NTM, and therefore find applications in the medical fields including hospitals, research institutes, etc.

### TECHNICAL FIELD

The present invention relates to the detection of Mycobacterium tuberculosis and nontuberculous mycobacteria. More particularly, the present invention relates to a primer set and/or a probe capable of detecting specific nucleotide sequences of Mycobacterium tuberculosis and nontuberculous mycobacteria, a kit for the detection of Mycobacterium tuberculosis and nontuberculous mycobacteria, comprising the same, and a method for detecting Mycobacterium tuberculosis and nontuberculous mycobacteria simultaneously, by duplex real-time PCR using the same.

### BACKGROUND ART

Nontuberculous mycobacteria are widely distributed in the environment, particularly in wet soil, marshland and rivers, and had been recognized as non-pathogenic bacteria before the discovery of its opportunistic characteristics. In the 1980s, nontuberculous mycobacteria were found to be an opportunistic pathogen of pulmonary diseases in patients with acquired immunodeficiency syndrome (AIDS). Further, the bacteria were also known to cause diseases in other patients. With the report of the pathogenesis of nontuberculous mycobacteria, its clinical significance has been increasingly recognized.

Recently, the United States of America and many European countries, which have a low prevalence of tuberculosis, have seen an increase in the incidence of infections caused by nontuberculous mycobacteria. In Korea, the isolation of nontuberculous mycobacteria from clinical specimens has also increased, although the incidence of tuberculosis has been greatly reduced. Thanks to a policy granting medical insurance for the performing of liquid culture of tuberculosis bacteria in 2009 in Korea, there was an increase in the number of laboratories designed to perform liquid culture. Liquid culture detects nontuberculous mycobacteria more often than does solid culture. Reports showed that nontuberculous mycobacteria was detected in about 12% of smear-positive tuberculosis cases as measured by liquid culture, with nontuberculous mycobacteria separated from the sputum accounting for about 10˜20% of pulmonary disease cases in Japan, Hong Kong, and Korea, and for about 40˜50% of pulmonary disease in the USA, Canada, and West Europe.

Resembling tuberculosis, which progresses slowly, the pulmonary diseases caused by nontuberculous mycobacteria are apt to be diagnosed wrongly. However, since drugs effective for the inhibition of Mycobacterium tuberculosis are different from those inhibitory of nontuberculous mycobacteria, there is a demand for rapid and accurate separation between tubercle bacilli and nontuberculous mycobacteria so that suitable drugs can be selected.

The conventional reagents in which the primer specific for the genus Mycobacterium is used as the detecting one for nontuberculous mycobacteria are problematic in terms of the accuracy. For example, when only a certain concentration of Mycobacterium tuberculosis is present, the reagents do not respond to primers for detecting Mycobacterium tuberculosis, but only to primers for nontuberculous mycobacteria, so Mycobacterium tuberculosis is wrongly identified as nontuberculous mycobacteria. On the other hand, when nontuberculous mycobacteria coexist with Mycobacterium tuberculosis, only nontuberculous mycobacteria are likely to be detected. Therefore, there is a need for a primer set and/or a probe capable of recognizing a nucleotide sequence that is absent from Mycobacterium tuberculosis, but intrinsic to nontuberculous mycobacteria, and for a method for separately detecting Mycobacterium tuberculosis and nontuberculous mycobacteria with rapidity and accuracy using the same.

### DISCLOSURE

### Technical Problem

It is an object of the present invention to provide a primer set specific for the IS6110 gene exclusive to Mycobacterium tuberculosis, and a primer set specific for the 16S rRNA of nontuberculous mycobacterium, both of which are applicable to the accurate detection and diagnosis of Mycobacterium tuberculosis and nontuberculous mycobacterium, separately.

It is another object of the present invention to provide probes for accurately detecting and diagnosing Mycobacterium tuberculosis and nontuberculous mycobacteria which are selectively detective of Mycobacterium tuberculosis-specific IS6110 gene or 16S rRNA and nontuberculous mycobacterium-specific 16S rRNA, respectively.

It is another object of the present invention to provide a kit for the detection of Mycobacterium tuberculosis and nontuberculous mycobacterium, comprising the primer sets and/or probes.

It is another object of the present invention to provide a method for the detection and diagnosis of Mycobacterium tuberculosis and nontuberculous mycobacteria with accuracy using a duplex real-time polymerase chain reaction based on the primer and/or probe.

### Technical Solution

In accordance with an aspect thereof, the present invention provides a probe for detecting the 16S rRNA gene of nontuberculous mycobacteria, having the nucleotide sequence of SEQ ID NO: 9.

In accordance with another aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; a probe, having the nucleotide sequence of SEQ ID NO: 3, for detecting the IS6110 gene of Mycobacterium tuberculosis; a primer set specific for the 16S rRNA gene of nontuberculous mycobacteria, composed of a forward primer having the nucleotide sequence of SEQ ID NO: 4, at least one reverse primer selected from the group consisting of nucleotide sequences of SEQ ID NOS: 5 to 7, and a reverse primer having the nucleotide sequence of SEQ ID NO: 8; and a probe, having the nucleotide sequence of SEQ ID NO: 9, for detecting the 16S rRNA gene of nontuberculous mycobacteria.

In accordance with a further aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 2, a probe, having the nucleotide sequence of SEQ ID NO: 3, for detecting the IS6110 gene of Mycobacterium tuberculosis, a primer set specific for the 16S rRNA gene of nontuberculous mycobacteria, composed of a forward primer having the nucleotide sequence of SEQ ID NO: 4, at least one reverse primer selected from the group consisting of nucleotide sequences of SEQ ID NOS: 5 to 7 and a reverse primer having the nucleotide sequence of SEQ ID NO: 8, and a probe, having the nucleotide sequence of SEQ ID NO: 9, for detecting the 16S rRNA gene of nontuberculous mycobacteria; and analyzing products of the duplex real-time PCR.

In accordance with a further aspect thereof, the present invention provides a primer set specific for the 16S rRNA gene of nontuberculous mycobacteria, comprising a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8.

In accordance with a still further aspect thereof, the present invention provides a probe for detecting the 16S rRNA gene of nontuberculous mycobacteria, having the nucleotide sequence of SEQ ID NO: 23.

In accordance with still another aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8; and a probe, having the nucleotide sequence of SEQ ID NO: 23, for detecting the 16S rRNA gene of nontuberculous mycobacteria.

In accordance with a yet further aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8; and a probe, having the nucleotide sequence of SEQ ID NO: 23, for detecting the 16S rRNA gene of nontuberculous mycobacteria; and analyzing products of the duplex real-time PCR.

In accordance with yet another aspect thereof, the present invention provides a probe for detecting the 16S rRNA gene of Mycobacterium tuberculosis, having the nucleotide sequence of SEQ ID NO: 26.

In accordance with a yet still further aspect thereof, the present invention provides a probe for detecting the 16S rRNA of nontuberculous mycobacterium, comprising a probe having the nucleotide sequence of SEQ ID NO: 27 and a probe having the nucleotide sequence of SEQ ID NO: 28.

In accordance with yet still another aspect thereof, the present invention provides a kit for the detection of Mycobacterium tuberculosis and nontuberculous mycobacteria, comprising: a universal primer set for amplifying 16S rRNA genes of Mycobacterium tuberculosis and nontuberculous mycobacterium, composed of a forward primer having the nucleotide sequence of SEQ ID NO: 24 and a reverse primer having the nucleotide sequence of SEQ ID NO: 25; a probe for detecting the 16S rRNA gene of Mycobacterium tuberculosis, having the nucleotide sequence of SEQ ID NO: 26; and a probe for detecting the 16S rRNA gene of nontuberculous mycobacteria, composed of a probe having the nucleotide sequence of SEQ ID NO: 27 and a probe having the nucleotide sequence of SEQ ID NO: 28.

In accordance with an additional aspect thereof, the present invention provides a method for detecting Mycobacterium tuberculosis and nontuberculous mycobacteria, comprising: isolating DNA from a test subject; amplifying the DNA by duplex real-time PCR using a universal primer set for amplifying 16S rRNA genes of Mycobacterium tuberculosis and nontuberculous mycobacterium, composed of a forward primer having the nucleotide sequence of SEQ ID NO: 24 and a reverse primer having the nucleotide sequence of SEQ ID NO: 25; a probe for detecting the 16S rRNA gene of Mycobacterium tuberculosis, having the nucleotide sequence of SEQ ID NO: 26; and a probe for detecting the 16S rRNA gene of nontuberculous mycobacteria, composed of a probe having the nucleotide sequence of SEQ ID NO: 27 and a probe having the nucleotide sequence of SEQ ID NO: 28.

In accordance with another additional aspect thereof, the present invention provides a probe for detecting the 16S rRNA gene of nontuberculous mycobacteria, having one selected from the group consisting of nucleotide sequences of SEQ ID NOS: 37 to 39.

In accordance with a further additional aspect thereof, the present invention provides a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 35, and a reverse primer having the nucleotide sequence of SEQ ID NO: 36; and a probe for detecting the 16S rRNA gene of nontuberculous mycobacteria, having one selected from the group consisting of nucleotides sequences of SEQ ID NOS: 37 to 39.

In accordance with a still additional aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20, a probe, having the nucleotide sequence of SEQ ID NO: 21, for detecting the IS6110 gene of Mycobacterium tuberculosis, a primer set specific for the 16S rRNA gene of nontuberculous mycobacteria, composed of a forward primer having the nucleotide sequence of SEQ ID NO: 35, and a reverse primer having the nucleotide sequence of SEQ ID NO: 36, and a probe for detecting the 16S rRNA gene of nontuberculous mycobacteria, having one selected from the group consisting of nucleotides sequences of SEQ ID NOS: 37 to 39; and analyzing products of the duplex real-time PCR.

In accordance with still another additional aspect thereof, the present invention provides a primer set specific for the 16S rRNA gene of nontuberculous mycobacteria, comprising a forward primer having the nucleotide sequence of SEQ ID NO: 61, and a reverse primer composed of a primer having the nucleotide sequence of SEQ ID NO: 5 and a primer having the nucleotide sequence of SEQ ID NO: 8.

In accordance with a still further additional aspect thereof, the present invention provides a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59; a probe for detecting the IS6110 gene of Mycobacterium tuberculosis, having the nucleotide sequence of SEQ ID NO: 21 or SEQ ID NO: 60; a primer set specific for the 16S rRNA gene of nontuberculous mycobacteria, comprising a forward primer having the nucleotide sequence of SEQ ID NO: 61, and a reverse primer composed of a primer having the nucleotide sequence of SEQ ID NO: 5 and a primer having the nucleotide sequence of SEQ ID NO: 8; and a probe for detecting the 16S rRNA gene of nontuberculous mycobacteria, having the nucleotide sequence of SEQ ID NO: 62 or 63.

In accordance with a yet additional aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59, a probe for detecting the IS6110 gene of Mycobacterium tuberculosis, having the nucleotide sequence of SEQ ID NO: 21 or SEQ ID NO: 60, a primer set specific for the 16S rRNA gene of nontuberculous mycobacteria, comprising a forward primer having the nucleotide sequence of SEQ ID NO: 61, and a reverse primer composed of a primer having the nucleotide sequence of SEQ ID NO: 5 and a primer having the nucleotide sequence of SEQ ID NO: 8; and a probe for detecting the 16S rRNA gene of nontuberculous mycobacteria, having the nucleotide sequence of SEQ ID NO: 62 or 63; and analyzing products of the duplex real-time PCR.

In accordance with yet another additional aspect thereof, the present invention provides a primer set specific for the 16S rRNA gene of nontuberculous mycobacteria, comprising: a forward primer comprising a primer having the nucleic acid sequence of SEQ ID NO: 65, a primer having the nucleotide sequence of SEQ ID NO: 66, and a primer having the nucleotide sequence of SEQ ID NO: 67; and a reverse primer comprising the nucleotide sequence of SEQ ID NO: 36.

In accordance with a yet further additional aspect thereof, the present invention provides a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 20; a probe for detecting the IS6110 gene of Mycobacterium tuberculosis, having the nucleotide sequence of SEQ ID NO: 21 or SEQ ID NO: 64; a primer set specific for the 16S rRNA gene of nontuberculous mycobacteria, composed of a forward primer comprising a primer having the nucleotide sequence of SEQ ID NO: 65, a primer having the nucleotide sequence of SEQ ID NO: 66, and a primer having the nucleotide sequence of SEQ ID NO: 67, and a reverse primer having the nucleotide sequence of SEQ ID NO: 36; and a probe for detecting the 16S rRNA gene of nontuberculous mycobacteria, having the nucleotide sequence of SEQ ID NO: 39 or 68.

In accordance with a still yet additional aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 20, a probe for detecting the IS6110 gene of Mycobacterium tuberculosis, having the nucleotide sequence of SEQ ID NO: 21 or SEQ ID NO: 64, a primer set specific for the 16S rRNA gene of nontuberculous mycobacteria, composed of a forward primer comprising a primer having the nucleotide sequence of SEQ ID NO: 65, a primer having the nucleotide sequence of SEQ ID NO: 66, and a primer having the nucleotide sequence of SEQ ID NO: 67, and a reverse primer having the nucleotide sequence of SEQ ID NO: 36, and a probe for detecting the 16S rRNA gene of nontuberculous mycobacteria, having the nucleotide sequence of SEQ ID NO: 39 or 68; and analyzing products of the duplex real-time PCR.

In accordance with still yet another additional aspect thereof, the present invention provides a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59; a probe for detecting the IS6110 gene of Mycobacterium tuberculosis, having the nucleotide sequence of SEQ ID NO: 60; a primer set specific for the 16S rRNA gene of nontuberculous mycobacteria, composed of a forward primer having the nucleotide sequence of SEQ ID NO: 24, and a reverse primer having the nucleotide sequence of SEQ ID NO: 75; and a probe for detecting the 16S rRNA gene of nontuberculous mycobacteria, comprising a probe having the nucleotide sequence of SEQ ID NO: 27 and a probe having the nucleotide sequence of SEQ ID NO: 76.

In accordance with a still yet further additional aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59, a probe for detecting the IS6110 gene of Mycobacterium tuberculosis, having the nucleotide sequence of SEQ ID NO: 60, a primer set specific for the 16S rRNA gene of nontuberculous mycobacteria, composed of a forward primer having the nucleotide sequence of SEQ ID NO: 24, and a reverse primer having the nucleotide sequence of SEQ ID NO: 75, and a probe for detecting the 16S rRNA gene of nontuberculous mycobacteria, comprising a probe having the nucleotide sequence of SEQ ID NO: 27 and a probe having the nucleotide sequence of SEQ ID NO: 76; and analyzing products of the duplex real-time PCR.

### Advantageous Effects

As described above, primer sets and/or probes for the detection of Mycobacterium tuberculosis and nontuberculous mycobacteria, capable of detecting nucleotide sequences characteristic of Mycobacterium tuberculosis and nontuberculous mycobacteria, detection kits, and duplex real-time PCR-based methods for detecting Mycobacterium tuberculosis and nontuberculous mycobacteria using the primer sets and/or probes, or the kits are provided by the present invention. Because the primers and/or probes are exclusive to Mycobacterium tuberculosis and nontuberculous mycobacteria, the methods of the present invention can be clinically applied to the detection of both Mycobacterium tuberculosis and nontuberculous mycobacteria at the same time, with higher efficiency.

### DESCRIPTION OF DRAWINGS

FIGS. 1 and 2 are graphs in which fluorescence intensities in green and yellow channels are plotted, respectively, against the number of cycles of real-time PCR with MTC.

FIGS. 3 and 4 are graphs in which fluorescence intensities in green and yellow channels are plotted, respectively, against the number of cycles of real-time PCR with NTM.

FIGS. 5 and 6 are graphs in which fluorescence intensities in green and yellow channels are plotted, respectively, against the number of cycles of real-time PCR with MTC+NTM.

FIGS. 7 and 8 are graphs in which fluorescence intensities in yellow and green channels are plotted, respectively, against the number of cycles of real-time PCR with MTC.

FIGS. 9 and 10 are graphs in which fluorescence intensities in yellow and green channels are plotted, respectively, against the number of cycles of real-time PCR with NTM.

FIGS. 11 and 12 are graphs in which fluorescence intensities in yellow and green channels are plotted, respectively, against the number of cycles of real-time PCR with MTC+NTM.

FIGS. 13 and 14 are graphs in which fluorescence intensities in green and yellow channels are plotted, respectively, against the number of cycles of real-time PCR with MTC.

FIGS. 15 and 16 are graphs in which fluorescence intensities in green and yellow channels are plotted, respectively, against the number of cycles of real-time PCR with NTM.

FIGS. 17 and 18 are graphs in which fluorescence intensities in green and yellow channels are plotted, respectively, against the number of cycles of real-time PCR with MTC+NTM.

FIGS. 19 and 20 are graphs in which fluorescence intensities in green and yellow channels are plotted, respectively, against the number of cycles of real-time PCR with MTC.

FIGS. 21 and 22 are graphs in which fluorescence intensities in green and yellow channels are plotted, respectively, against the number of cycles of real-time PCR with NTM.

FIGS. 23 and 24 are graphs in which fluorescence intensities in green and yellow channels are plotted, respectively, against the number of cycles of real-time PCR with MTC+NTM.

FIGS. 25 and 26 are graphs in which fluorescence intensities in green and yellow channels are plotted, respectively, against the number of cycles of real-time PCR with MTC.

FIGS. 27 and 28 are graphs in which fluorescence intensities in green and yellow channels are plotted, respectively, against the number of cycles of real-time PCR with NTM.

FIGS. 29 and 30 are graphs in which fluorescence intensities in green and yellow channels are plotted, respectively, against the number of cycles of real-time PCR with MTC+NTM.

FIGS. 31 and 32 are graphs in which fluorescence intensities in green and yellow channels are plotted, respectively, against the number of cycles of real-time PCR with MTC.

FIGS. 33 and 34 are graphs in which fluorescence intensities in green and yellow channels are plotted, respectively, against the number of cycles of real-time PCR with NTM.

FIGS. 35 and 36 are graphs in which fluorescence intensities in green and yellow channels are plotted, respectively, against the number of cycles of real-time PCR with MTC+NTM.

FIGS. 37 and 38 are graphs in which fluorescence intensities in green and yellow channels are plotted, respectively, against the number of cycles of real-time PCR with MTC.

FIGS. 39 and 40 are graphs in which fluorescence intensities in green and yellow channels are plotted, respectively, against the number of cycles of real-time PCR with NTM.

FIGS. 41 and 42 are graphs in which fluorescence intensities in green and yellow channels are plotted, respectively, against the number of cycles of real-time PCR with MTC+NTM.

### BEST MODE

According to an aspect thereof, the present invention addresses a probe for detecting the 16S rRNA gene of nontuberculous mycobacteria, having the nucleotide sequence of SEQ ID NO: 9.

In one embodiment, the probe is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ (molecular grove binding non-fluorescence quencher).

For use in the detection of the 16S rRNA gene of nontuberculous mycobacteria, the probe having the nucleotide sequence of SEQ ID NO: 9 is specific for the product obtained by carrying out a polymerase chain reaction in the presence of a primer set specific for the 16S rRNA gene of nontuberculous mycobacteria which comprises a forward primer having the nucleotide sequence of SEQ ID NO: 4; at least one reverse primer selected from the group consisting of a primer having the nucleotide sequence of SEQ ID NO: 5, a primer having the nucleotide sequence of SEQ ID NO: 6, and a primer having the nucleotide sequence of SEQ ID NO: 7; and a reverse primer having the nucleotide sequence of SEQ ID NO: 8.

To this end, the present invention envisages a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; a probe for detecting the IS6110 gene of Mycobacterium tuberculosis, having the nucleotide sequence of SEQ ID NO: 3; a primer set specific for the 16S rRNA gene of nontuberculous mycobacteria, composed of a forward primer having the nucleotide sequence of SEQ ID NO: 4, at least one reverse primer selected from the group consisting of nucleotide sequences of SEQ ID NOS: 5 to 7, and a reverse primer having the nucleotide sequence of SEQ ID NO: 8; and a probe for detecting the 16S rRNA gene of nontuberculous mycobacteria, having the nucleotide sequence of SEQ ID NO: 9.

The detection kit may further comprise a reagent necessary for amplifying DNA by PCR. This reagent may include DNA polymerase, dNTPs, PCR buffer, etc.

The probe for detecting the 16S rRNA gene of nontuberculous mycobacteria, having the nucleotide sequence of SEQ ID NO: 9, and the probe for detecting the IS6110 gene of Mycobacterium tuberculosis, having the nucleotide sequence of SEQ ID NO: 3 may be labeled with different detectable means. This detectable means refers to compounds, biomolecules or biomimetics that can be conjugated, connected, or attached to probes to provide quantitative indices such as density, concentration, quantity, etc. Examples of the detectable means include fluorescent markers, luminescents, bioluminescents, and radio isotopes, but are not limited thereto. Account must be taken of whether fluorescent markers can be separately detected when they are used together in a PCR reaction because the usages of the fluorescent markers differ from one to another if the fluorescent markers are different in excitation and emission wavelengths according to their kind. If used together, two or more fluorescent markers may be different in color. Details and selection of the fluorescent markers are obvious to those skilled in the art.

The kit for the detection of Mycobacterium tuberculosis and nontuberculous mycobacteria comprises a primer set, specific for the IS6110 of Mycobacterium tuberculosis, which consists of a forward primer and a reverse primer having the nucleotide sequences of SEQ ID NOS:1 and 2, respectively. The primer set specific for the IS6100 gene is designed to detect all of various mycobacterium species (Mycobacterium tuberculosis complex, MTC).

In one embodiment of the present invention, the respective probes for detecting the IS6110 gene of Mycobacterium tuberculosis and the 16S rRNA of nontuberculous mycobacteria, having the nucleotide sequences of SEQ ID NOS: 3 and 9, may be Taqman probes.

The probe for detecting the IS6110 gene of Mycobacterium tuberculosis, having the nucleotide sequence of SEQ ID NO: 3, is specific for the product obtained by carrying out PCR in the presence of a primer set specific for the IS6110 gene of Mycobacterium tuberculosis which comprises a forward primer and a reverse primer having nucleotides sequences of SEQ ID NOS: 1 and 2, respectively.

In accordance with one embodiment of the present invention, the forward primer having the nucleotide sequence of SEQ ID NO: 4 is specific for the 16S rRNA gene of nontuberculous mycobacteria. The forward primer of SEQ ID NO: 4 (5′-ggyrayctgccctgcac-3′) may be a primer set comprising a group of the primers 5′-ggtaatctgccctgcac-3′ (SEQ ID NO: 12), 5′-ggtaacctgccctgcac-3′ (SEQ ID NO: 13), 5′-ggcaatctgccctgcac-3′ (SEQ ID NO: 14), 5′-ggcaacctgccctgcac-3′ (SEQ ID NO: 15), 5′-ggtgatctgccctgcac-3′ (SEQ ID NO: 16), 5′-ggtgacctgccctgcac-3′ (SEQ ID NO: 17), 5′-ggcgatctgccctgcac-3′ (SEQ ID NO: 18), and 5′-ggcgacctgccctgcac-3′ (SEQ ID NO: 19). For example, it is a primer set in which 5′-ggtaatctgccctgcac-3′, 5′-ggtaacctgccctgcac-3′, 5′-ggcaatctgccctgcac-3′, 5′-ggcaacctgccctgcac-3′, 5′-ggtgatctgccctgcac-3′, 5′-ggtgacctgccctgcac-3′, 5′-ggcgatctgccctgcac-3′, and 5′-ggcgacctgccctgcac-3′ are mixed at a ratio of approximately 1:1:1:1:1:1:1:1.

All of the nucleotide sequences of SEQ ID NOS: 5, 6 and 7 (NTM-1) are reverse primers specific for the 16S rRNA. The nucleotide sequence of SEQ ID NO: 8 (NTM-2) is a reverse primer specific for the 16S rRNA gene of nontuberculous mycobacteria. All of the reverse primers for the 16S rRNA gene of nontuberculous mycobacteria are designed to detect all of various nontuberculous mycobacterium species. The reverse primer of SEQ ID NO: 8 (5′-catcccacaccgctaccw-3′) refers to a primer set comprising 5′-catcccacaccgctacct-3′ (SEQ ID NO: 10) and 5′-catcccacaccgctacca-3′ (SEQ ID NO: 11). For example, the nucleotide sequence of SEQ ID NO: 8 may be a primer set in which 5′-catcccacaccgctacct-3′ and 5′-catcccacaccgctacca-3′ are mixed at a ratio of approximately 1:1.

In another embodiment of the present invention, the probe for detecting the IS6110 gene of Mycobacterium tuberculosis and the probe for detecting the 16S rRNA gene of nontuberculous mycobacteria are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA, BHQ-1,2,3 and MGBNFQ (molecular grove binding non-fluorescence quencher). The fluorescent markers labeled at the 5′ ends may differ from the probe for detecting the IS61110 gene of Mycobacterium tuberculosis to the probe for detecting the 16S rRNA of nontuberculous mycobacteria. For instance, the probe for detecting the IS6110 gene of Mycobacterium tuberculosis may be labeled at the 5′ end with VIC and at the 3′ end with MGBNFQ while the probe for detecting the 16S rRNA gene of nontuberculous mycobacteria may be labeled at the 5′ end with FAM and at the 3′ end with MGBNFQ.

In one embodiment, the kit for the detection of Mycobacterium tuberculosis and nontuberculous mycobacteria may comprise the reverse primers having the nucleotide sequences of SEQ ID NOS: 5 and 8 at a ratio of 1:1, the latter being composed of SEQ ID NOS: 10 and 11 at a ratio of 1:1. Accordingly, in the kit, the primer of SEQ ID NO: 5, 5′-catcccacaccgctacct-3′, and 5′-catcccacaccgctacca-3′ may be mixed at a ratio of 2:1:1.

In another embodiment of the present invention, the kit for the detection of Mycobacterium tuberculosis and nontuberculous mycobacteria may comprise the reverse primers having the nucleotide sequences of SEQ ID NOS: 6 and 8 at a ratio of 1:1, the latter being composed of SEQ ID NOS: 10 and 11 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 7 and 8 at a ratio of 1:1, the latter being composed of SEQ ID NOS: 10 and 11 at a ratio of 1:1.

In accordance with a further aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 2, a probe, having the nucleotide sequence of SEQ ID NO: 3, for detecting the IS6110 gene of Mycobacterium tuberculosis, a primer set specific for the 16S rRNA gene of nontuberculous mycobacteria, composed of a forward primer having the nucleotide sequence of SEQ ID NO: 4, at least one reverse primer (NTM-1) selected from the group consisting of nucleotide sequences of SEQ ID NOS: 5 to 7 and a reverse primer (NTM-2) having the nucleotide sequence of SEQ ID NO: 8, and a probe, having the nucleotide sequence of SEQ ID NO: 9, for detecting the 16S rRNA gene of nontuberculous mycobacteria; and analyzing products of the duplex real-time PCR.

In accordance with a further aspect thereof, the present invention addresses a primer set specific for the 16S rRNA gene of nontuberculous mycobacteria, comprising a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8.

The forward primer having the nucleotide sequence of SEQ ID NO: 22 is specific for the 16S rRNA gene of nontuberculous mycobacteria.

All of the nucleotide sequences of SEQ ID NOS: 5, 6 and 7 (NTM-1) are used as reverse primers specific for the 16S rRNA. The nucleotide sequence of SEQ ID NO: 8 (NTM-2) is a reverse primer specific for the 16S rRNA gene of nontuberculous mycobacteria. All of the reverse primers for the 16S rRNA gene of nontuberculous mycobacteria are designed to detect all of various nontuberculous mycobacterium species. The reverse primer of SEQ ID NO: 8 (5′-catcccacaccgctaccw-3′) refers to a primer set comprising 5′-catcccacaccgctacct-3′ (SEQ ID NO: 10) and 5′-catcccacaccgctacca-3′ (SEQ ID NO: 11), for example at a ratio of approximately 1:1.

Also, contemplated in accordance with a still further aspect of the present invention is a probe for detecting the 16S rRNA gene of nontuberculous mycobacteria, having the nucleotide sequence of SEQ ID NO: 23.

In one embodiment, the probe is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, and BHQ-1,2,3.

The probe for detecting the 16S rRNA gene of nontuberculous mycobacteria, having the nucleotide sequence of SEQ ID NO: 23, is specific for the product obtained by carrying out a polymerase chain reaction in the presence of a primer set specific for the 16S rRNA gene of nontuberculous mycobacteria which comprises a forward primer having the nucleotide sequence of SEQ ID NO: 22; at least one reverse primer selected from the group consisting of a primer having the nucleotide sequence of SEQ ID NO: 5, a primer having the nucleotide sequence of SEQ ID NO: 6, and a primer having the nucleotide sequence of SEQ ID NO: 7; and a reverse primer having the nucleotide sequence of SEQ ID NO: 8.

In accordance with still another aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8; and a probe, having the nucleotide sequence of SEQ ID NO: 23, for detecting the 16S rRNA gene of nontuberculous mycobacteria.

The detection kit may further comprise a reagent necessary for amplifying DNA by PCR. This reagent may include DNA polymerase, dNTPs, PCR buffer, etc.

The probe for detecting the 16S rRNA gene of nontuberculous mycobacteria, having the nucleotide sequence of SEQ ID NO: 23, and the probe for detecting the IS6110 gene of Mycobacterium tuberculosis, having the nucleotide sequence of SEQ ID NO: 21 may be labeled with different detectable means. This detectable means refers to compounds, biomolecules or biomimetics that can be conjugated, connected, or attached to probes to provide quantitative indices such as density, concentration, quantity, etc. Examples of the detectable means include fluorescent markers, luminescents, bioluminescents, and radio isotopes, but are not limited thereto. Account must be taken of whether fluorescent markers can be separately detected when they are used together in a PCR reaction because the usages of the fluorescent markers differ from one to another if the fluorescent markers are different in excitation and emission wavelength according to their kind. If used together, two or more fluorescent markers may be different in color. Details and selection of the fluorescent markers are obvious to those skilled in the art.

The kit for the detection of Mycobacterium tuberculosis and nontuberculous mycobacteria comprises a primer set, specific for the IS6110 of Mycobacterium tuberculosis, which consists of a forward primer and a reverse primer having the nucleotide sequences of SEQ ID NOS: 1 and 20, respectively. The primer set specific for the IS6100 gene is designed to detect all of various mycobacterium species (Mycobacterium tuberculosis complex, MTC).

In one embodiment of the present invention, the respective probes for detecting the IS6110 gene of Mycobacterium tuberculosis and the 16S rRNA of nontuberculous mycobacteria, having the nucleotide sequences of SEQ ID NOS: 21 and 23, may be Taqman probes.

The probe for detecting the IS6110 gene of Mycobacterium tuberculosis, having the nucleotide sequence of SEQ ID NO: 21, is specific for the product obtained by carrying out PCR in the presence of a primer set specific for the IS6110 gene of Mycobacterium tuberculosis which comprises a forward primer and a reverse primer having nucleotides sequences of SEQ ID NOS: 1 and 20, respectively.

All of the nucleotide sequences of SEQ ID NOS: 5, 6 and 7 (NTM-1) are reverse primers specific for the 16S rRNA. The nucleotide sequence of SEQ ID NO: 8 (NTM-2) is a reverse primer specific for the 16S rRNA gene of nontuberculous mycobacteria. All of the reverse primers for the 16S rRNA gene of nontuberculous mycobacteria are designed to detect all of various nontuberculous mycobacterium species. The reverse primer of SEQ ID NO: 8 (5′-catcccacaccgctaccw-3′) refers to a primer set comprising 5′-catcccacaccgctacct-3′ (SEQ ID NO: 10) and 5′-catcccacaccgctacca-3′ (SEQ ID NO: 11). For example, the nucleotide sequence of SEQ ID NO: 8 may be a primer set in which 5′-catcccacaccgctacct-3′ and 5′-catcccacaccgctacca-3′ are mixed at a ratio of approximately 1:1.

In another embodiment of the present invention, the probe for detecting the IS6110 gene of Mycobacterium tuberculosis and the probe for detecting the 16S rRNA gene of nontuberculous mycobacteria are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA, BHQ-1,2,3 and MGBNFQ (molecular grove binding non-fluorescence quencher). The fluorescent markers labeled at the 5′ ends may differ from the probe for detecting the IS61110 gene of Mycobacterium tuberculosis to the probe for detecting the 16S rRNA of nontuberculous mycobacteria. For instance, the probe for detecting the IS6110 gene of Mycobacterium tuberculosis may be labeled at the 5′ end with HEX and at the 3′ end with BHQ-1 while the probe for detecting the 16S rRNA gene of nontuberculous mycobacteria may be labeled at the 5′ end with FAM and at the 3′ end with BHQ-1.

In one embodiment, the kit for the detection of Mycobacterium tuberculosis and nontuberculous mycobacteria may comprise the reverse primers having the nucleotide sequences of SEQ ID NOS: 5 and 8 at a ratio of 1:1, the latter being composed of SEQ ID NOS: 10 and 11 at a ratio of 1:1. Accordingly, in the kit, the primer of SEQ ID NO: 5, 5′-catcccacaccgctacct-3′, and 5′-catcccacaccgctacca-3′ may be mixed at a ratio of 2:1:1.

In another embodiment of the present invention, the kit for the detection of Mycobacterium tuberculosis and nontuberculous mycobacteria may comprise the reverse primers having the nucleotide sequences of SEQ ID NOS: 6 and 8 at a ratio of 1:1, the latter being composed of SEQ ID NOS: 10 and 11 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 7 and 8 at a ratio of 1:1, the latter being composed of SEQ ID NOS: 10 and 11 at a ratio of 1:1.

In accordance with a yet further aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 22, at least one reverse primer selected from the group consisting of nucleotide sequences of SEQ ID NOS: 5 to 7, and a reverse primer having the nucleotide sequence of SEQ ID NO: 8; and a probe, having the nucleotide sequence of SEQ ID NO: 23, for detecting the 16S rRNA gene of nontuberculous mycobacteria; and analyzing products of the duplex real-time PCR.

In accordance with yet another aspect thereof, the present invention addresses a probe for detecting the 16S rRNA gene of Mycobacterium tuberculosis, having the nucleotide sequence of SEQ ID NO: 26.

In one embodiment of the present invention, the probe is labeled at the 5′ end with a fluorescent marker selected from the group consisting of FAM, VIC, TET, JOE, HEX, CY3, CY5, ROX, RED610, TEXAS RED, RED670, and NED, and at the 3′ ends with a fluorescence quencher selected from the group consisting of 6-TAMRA, BHQ-1,2,3 and MGBNFQ.

For use in the detection of the 16S rRNA gene of nontuberculous mycobacteria, the probe having the nucleotide sequence of SEQ ID NO: 26 is specific for nontuberculous mycobacterium-pertinent products obtained by carrying out a polymerase chain reaction in the presence of a universal primer specific for the 16S rRNA gene of Mycobacterium tuberculosis and nontuberculous mycobacteria which comprises a forward primer having the nucleotide sequence of SEQ ID NO: 24 and a reverse primer having the nucleotide sequence of SEQ ID NO: 25.

In accordance with a yet still further aspect thereof, the present invention addresses a probe for detecting the 16S rRNA of nontuberculous mycobacterium, comprising a probe having the nucleotide sequence of SEQ ID NO: 27 and a probe having the nucleotide sequence of SEQ ID NO: 28.

For use in the detection of the 16S rRNA gene of nontuberculous mycobacteria, the respective probes having the nucleotide sequences of SEQ ID NOS: 27 and 28 are specific for nontuberculous mycobacterium-pertinent products obtained by carrying out a polymerase chain reaction in the presence of a universal primer specific for the 16S rRNA gene of Mycobacterium tuberculosis and nontuberculous mycobacteria which comprises a forward primer having the nucleotide sequence of SEQ ID NO: 24, and a reverse primer having the nucleotide sequence of SEQ ID NO: 25.

In one embodiment of the present invention, the probe for detecting the IS6110 gene of Mycobacterium tuberculosis and the probe for detecting the 16S rRNA gene of nontuberculous mycobacteria are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA, BHQ-1,2,3 and MGBNFQ.

In accordance with yet still another aspect thereof, the present invention provides a kit for the detection of Mycobacterium tuberculosis and nontuberculous mycobacteria, comprising: a universal primer set for amplifying 16S rRNA genes of Mycobacterium tuberculosis and nontuberculous mycobacterium, composed of a forward primer having the nucleotide sequence of SEQ ID NO: 24 and a reverse primer having the nucleotide sequence of SEQ ID NO: 25; a probe for detecting the 16S rRNA gene of Mycobacterium tuberculosis, having the nucleotide sequence of SEQ ID NO: 26; and a probe for detecting the 16S rRNA gene of nontuberculous mycobacteria, composed of a probe having the nucleotide sequence of SEQ ID NO: 27 and a probe having the nucleotide sequence of SEQ ID NO: 28.

The detection kit may further comprise a reagent necessary for amplifying DNA by PCR. This reagent may include DNA polymerase, dNTPs, PCR buffer, etc.

The probe for detecting the 16S rRNA gene of nontuberculous mycobacteria, having the nucleotide sequence of SEQ ID NO: 26, and the probe for detecting the 16S rRNA gene of nontuberculous mycobacteria, composed of a probe having the nucleotide sequence of SEQ ID NO: 27 and a probe having the nucleotide sequence of SEQ ID NO: 28 may be labeled with different detectable means. This detectable means refers to compounds, biomolecules or biomimetics that can be conjugated, connected, or attached to probes to provide quantitative indices such as density, concentration, quantity, etc. Examples of the detectable means include fluorescent markers, luminescents, bioluminescents, and radio isotopes, but are not limited thereto. Account must be taken of whether fluorescent markers can be separately detected when they are used together in a PCR reaction because the usages of the fluorescent markers differ from one to another if the fluorescent markers are different in excitation and emission wavelength according to their kind. If used together, two or more fluorescent markers may be different in color. Details and selection of the fluorescent markers are obvious to those skilled in the art.

The forward primer having the nucleotide sequence of SEQ ID NO: 24 and the reverse primer having the nucleotide sequence of SEQ ID NO: 25 form a primer set, together, which can be universally used to amplify the 16S rRNA genes of mycobacteria including both MTC and NTM.

In one embodiment of the present invention, the forward primer having the nucleotide sequence of SEQ ID NO: 24 refers to a primer set comprising 5′-ggataagcctgggaaactgg-3′ (SEQ ID NO: 29) and 5′-ggataagcttgggaaactgg-3′ (SEQ ID NO: 30) at a ratio of approximately 1:1. In addition, the reverse primer having the nucleotide sequence of SEQ ID NO: 25 may be a primer set in which 5′-accccaccaacaagctgata-3′ (SEQ ID NO: 31) and 5′-accccaccaactagctgata-3′ (SEQ ID NO: 32) are mixed at a ratio of approximately 1:1.

In one embodiment of the present invention, the probe for detecting the 16s rRNA gene of Mycobacterium tuberculosis, having the nucleotide sequence of SEQ ID NO: 26, and the respective probes for detecting the 16S rRNA of nontuberculous mycobacteria, having the nucleotide sequences of SEQ ID NOS: 27 and 28, may be Taqman probes.

The probe for detecting the 16s rRNA gene of Mycobacterium tuberculosis, having the nucleotide sequence of SEQ ID NO: 26, and the respective probes for detecting the 16S rRNA of nontuberculous mycobacteria, having the nucleotide sequences of SEQ ID NOS: 27 and 28, are designed to target 16S rRNA regions.

In another embodiment, the NTM-2 probe having the nucleotide sequence of SEQ ID NO: 28 may contain 5′-FAM-tggaaagcgtttggtagc-MGB-3′ (SEQ ID NO: 33) and 5′-FAM-tggaaagtgtttggtagc-MGB-3′ (SEQ ID NO: 34) at a ratio of approximately 1:1.

In another embodiment of the present invention, the probe for detecting the IS6110 gene of Mycobacterium tuberculosis, having the nucleotide sequence of SEQ ID NO: 26, and the respective probes for detecting the 16S rRNA gene of nontuberculous mycobacteria, having the nucleotide sequences of SEQ ID NOS: 27 and 28, are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA and BHQ-1,2,3. The fluorescent markers labeled at the 5′ ends may differ from the probe for detecting the IS6110 gene of Mycobacterium tuberculosis, having the nucleotide sequence of SEQ ID NO: 26, and the respective probes for detecting the 16S rRNA gene of nontuberculous mycobacteria, having the nucleotide sequences of SEQ ID NOS: 27 and 28. For instance, the probe for detecting the IS6110 gene of Mycobacterium tuberculosis may be labeled at the 5′ end with VIC and at the 3′ end with MGBNFQ while the probe for detecting the 16S rRNA gene of nontuberculous mycobacteria may be labeled at the 5′ end with FAM and at the 3′ end with MAGBNFQ.

In accordance with an additional aspect thereof, the present invention addresses a method for detecting Mycobacterium tuberculosis and nontuberculous mycobacteria, comprising: isolating DNA from a test subject; amplifying the DNA by duplex real-time PCR using a universal primer set for amplifying 16S rRNA genes of Mycobacterium tuberculosis and nontuberculous mycobacterium, composed of a forward primer having the nucleotide sequence of SEQ ID NO: 24 and a reverse primer having the nucleotide sequence of SEQ ID NO: 25; a probe for detecting the 16S rRNA gene of Mycobacterium tuberculosis, having the nucleotide sequence of SEQ ID NO: 26; and a probe for detecting the 16S rRNA gene of nontuberculous mycobacteria, composed of a probe having the nucleotide sequence of SEQ ID NO: 27 and a probe having the nucleotide sequence of SEQ ID NO: 28.

In accordance with another additional aspect thereof, the present invention addresses a probe for detecting the 16S rRNA gene of nontuberculous mycobacteria, having one selected from the group consisting of nucleotide sequences of SEQ ID NOS: 37 to 39.

In another embodiment of the present invention, the probe for detecting the IS6110 gene of Mycobacterium tuberculosis and the probe for detecting the 16S rRNA gene of nontuberculous mycobacteria are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA, BHQ-1,2,3 and MGBNFQ (molecular grove binding non-fluorescence quencher).

For use in the detection of the 16S rRNA gene of nontuberculous mycobacteria, the probe having one selected from the group consisting of nucleotide sequences of SEQ ID NOS: 37 to 39 is specific for the product obtained by carrying out a polymerase chain reaction in the presence of a primer set specific for the 16S rRNA gene of nontuberculous mycobacteria which comprises a forward primer having the nucleotide sequence of SEQ ID NO: 35, and a reverse primer having the nucleotide sequence of SEQ ID NO: 36.

In accordance with a further additional aspect thereof, the present invention addresses a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20; a probe, having the nucleotide sequence of SEQ ID NO: 21, for detecting the IS6110 gene of Mycobacterium tuberculosis; a primer set specific for the 16S rRNA gene of nontuberculous mycobacteria, composed of a forward primer having the nucleotide sequence of SEQ ID NO: 35, and a reverse primer having the nucleotide sequence of SEQ ID NO: 36; and a probe for detecting the 16S rRNA gene of nontuberculous mycobacteria, having one selected from the group consisting of nucleotides sequences of SEQ ID NOS: 37 to 39.

The detection kit may further comprise a reagent necessary for amplifying DNA by PCR. This reagent may include DNA polymerase, dNTPs, PCR buffer, etc.

The probe for detecting the 16S rRNA gene of nontuberculous mycobacteria, having one selected from the group consisting of nucleotide sequences of SEQ ID NOS: 37 to 39, and the probe for detecting the IS6110 gene of Mycobacterium tuberculosis, having the nucleotide sequence of SEQ ID NO: 21, may be labeled with different detectable means. This detectable means refers to compounds, biomolecules or biomimetics that can be conjugated, connected, or attached to probes to provide quantitative indices such as density, concentration, quantity, etc. Examples of the detectable means include fluorescent markers, luminescents, bioluminescents, and radio isotopes, but are not limited thereto. Account must be taken of whether fluorescent markers can be separately detected when they are used together in a PCR reaction because the usages of the fluorescent markers differ from one to another if the fluorescent markers are different in excitation and emission wavelength according to their kind. If used together, two or more fluorescent markers may be different in color. Details and selection of the fluorescent markers are obvious to those skilled in the art.

As described above, the kit for the detection of Mycobacterium tuberculosis and nontuberculous mycobacteria comprises a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20. The primer set specific for the IS6100 gene of Mycobacterium tuberculosis is designed to detect all of various mycobacterium species (Mycobacterium tuberculosis complex, MTC).

In one embodiment of the present invention, the probe, having the nucleotide sequence of SEQ ID NO: 21, for detecting the IS6110 gene of Mycobacterium tuberculosis, and the probe, having one selected from the group consisting of the nucleotide sequences of SEQ ID NOS: 37 to 39, for detecting the 16S rRNA of nontuberculous mycobacteria, may be Taqman probes.

For use in the detection of the IS6110 gene of Mycobacterium tuberculosis, the probe having the nucleotide sequence of SEQ ID NO: 21 is specific for the product obtained by carrying out a polymerase chain reaction in the presence of a primer set specific for the IS6110 gene of Mycobacterium tuberculosis which comprises a forward primer having the nucleotide sequence of SEQ ID NO: 1, and a reverse primer having the nucleotide sequence of SEQ ID NO: 20.

For use in the detection of the 16S rRNA gene of nontuberculous mycobacteria, the probe having one selected from the group consisting of nucleotide sequences of SEQ ID NOS: 37 to 39 is specific for the product obtained by carrying out a polymerase chain reaction in the presence of a primer set specific for the 16S rRNA gene of nontuberculous mycobacteria which comprises a forward primer having the nucleotide sequence of SEQ ID NO: 35, and a reverse primer having the nucleotide sequence of SEQ ID NO: 36.

The nucleotide sequence of SEQ ID NO: 35 can be used as a forward primer specific for the 16S rRNA gene of nontuberculous mycobacteria. The forward primer specific for the 16S rRNA gene of nontuberculous mycobacteria is designed to detect all of various nontubrculous mycobacterium species. The nucleotide sequence of SEQ ID NO: 35 is a primer set comporising 5′-catgtcttgtgggggaaagctt-3′ (SEQ ID NO: 40), 5′-catgttttgtgggggaaagctt-3′ (SEQ ID NO: 41), 5′-catgtcttctgggggaaagctt-3′ (SEQ ID NO: 42), 5′-catgtcttgtggtggaaagctt-3′ (SEQ ID NO: 43), 5′-catgtcttgtggggcaaagctt-3′ (SEQ ID NO: 44), 5′-catgttttctgggggaaagctt-3′ (SEQ ID NO: 45), 5′-catgtcttctggtggaaagctt-3′ (SEQ ID NO: 46), 5′-catgtcttgtggtgcaaagctt-3′ (SEQ ID NO: 47), 5′-catgttttgtggggcaaagctt-3′ (SEQ ID NO: 48), 5′-catgtcttctggggcaaagctt-3′ (SEQ ID NO: 49), 5′-catgttttgtggtggaaagctt-3′ (SEQ ID NO: 50), 5′-catgttttctggtggaaagctt-3′ (SEQ ID NO: 51), 5′-catgttttctggggcaaagctt-3′ (SEQ ID NO: 52), 5′-catgttttgtggtgcaaagctt-3′ (SEQ ID NO: 53), 5′-catgtcttctggtgcaaagctt-3′ (SEQ ID NO: 54), and 5′-catgttttctggtgcaaagctt-3′ (SEQ ID NO: 55). In the primer set having the nucleotide sequence of SEQ ID NO: 35, for instance, the respective primers having the nucleotide sequences of SEQ ID NOS: 40 to 55 may be present in substantially the same amounts.

In another embodiment of the present invention, the probe for detecting the IS6110 gene of Mycobacterium tuberculosis and the probe for detecting the 16S rRNA gene of nontuberculous mycobacteria are labeled at their 5′ ends with a fluorescent marker selected from the group consisting of FAM, VIC, TET, JOE, HEX, CY3, CY5, ROX, RED610, TEXAS RED, RED670, and NED, and at their 3′ ends with a fluorescence quencher selected from the group consisting of 6-TAMRA and BHQ-1,2,3. The fluorescent markers labeled at the 5′ ends may differ from the probe for detecting the IS61110 gene of Mycobacterium tuberculosis to the probe for detecting the 16S rRNA of nontuberculous mycobacteria. For instance, the probe for detecting the IS6110 gene of Mycobacterium tuberculosis may be labeled at the 5′ end with HEX and at the 3′ end with BHQ-1 while the probe for detecting the 16S rRNA gene of nontuberculous mycobacteria may be labeled at the 5′ end with FAM and at the 3′ end with BHQ-1.

According to one embodiment, the probe for detecting 16S rRNA gene, having the nucleotide sequence of SEQ ID NO: 38 may be composed of FAM-cctgagagggtgaccgg-BHQ1 (SEQ ID NO: 56) and FAM-cctgagagggtgtccgg-BHQ1 (SEQ ID NO: 57) at a ratio of approximately 1:1 in the kit for the detection of Mycobacterium tuberculosis and nontuberculous mycobacteria.

In accordance with a still additional aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 1 and a reverse primer having the nucleotide sequence of SEQ ID NO: 20, a probe, having the nucleotide sequence of SEQ ID NO: 21, for detecting the IS6110 gene of Mycobacterium tuberculosis, a primer set specific for the 16S rRNA gene of nontuberculous mycobacteria, composed of a forward primer having the nucleotide sequence of SEQ ID NO: 35, and a reverse primer having the nucleotide sequence of SEQ ID NO: 36, and a probe for detecting the 16S rRNA gene of nontuberculous mycobacteria, having one selected from the group consisting of nucleotides sequences of SEQ ID NOS: 37 to 39; and analyzing products of the duplex real-time PCR.

In accordance with still another additional aspect thereof, the present invention addresses a primer set specific for the 16S rRNA gene of nontuberculous mycobacteria, comprising a forward primer having the nucleotide sequence of SEQ ID NO: 61, and a reverse primer composed of a primer having the nucleotide sequence of SEQ ID NO: 5 and a primer having the nucleotide sequence of SEQ ID NO: 8.

The nucleotide sequence of SEQ ID NO: 61 may be used as a forward primer specific for the 16S rRNA of nontuberculous mycobacteria.

The respective nucleotide sequences of SEQ ID NOS: 5 and 8 (NTM-1 and NTM-2) can be used as reverse primers specific for the 16S rRNA gene. The reverse primer specific for the 16S rRNA gene is designed to detect all of various nontuberculous mycobacterium species. The nucleotide sequence of SEQ ID NO: 8 (5′-catcccacaccgctaccw-3′) is a primer set comprising 5′-catcccacaccgctacct-3′ (SEQ ID NO: 10) and 5′-catcccacaccgctacca-3′ (SEQ ID NO: 11) at a ratio of approximately 1:1.

In accordance with a still further additional aspect thereof, the present invention addresses a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59; a probe for detecting the IS6110 gene of Mycobacterium tuberculosis, having the nucleotide sequence of SEQ ID NO: 21 or SEQ ID NO: 60; a primer set specific for the 16S rRNA gene of nontuberculous mycobacteria, comprising a forward primer having the nucleotide sequence of SEQ ID NO: 61, and a reverse primer composed of a primer having the nucltotide sequence of SEQ ID NO: 5 and a primer having the nucleotide sequence of SEQ ID NO: 8; and a probe for detecting the 16S rRNA gene of nontuberculous mycobacteria, having the nucleotide sequence of SEQ ID NO: 62 or 63.

The detection kit may further comprise a reagent necessary for amplifying DNA by PCR. This reagent may include DNA polymerase, dNTPs, PCR buffer, etc.

According to one embodiment of the present invention, the probe for detecting the IS6110 gene of Mycobacterium tuberculosis, having the nucleotide sequence of SEQ ID NO: 21 or 60, and the probe for detecting the 16S rRNA gene of nontuberculous mycobacteria, having the nucleotide sequence of SEQ ID NO: 62 or 63, may be labeled with different detectable means.

In the kit for the detection of Mycobacterium tuberculosis and nontuberculous mycobacteria, comprising: a primer set specific for the IS6110 gene of Mycobacterium tuberculosis, as described above, a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59 is employed. The primer set specific for the IS6100 gene of Mycobacterium tuberculosis is designed to detect all of various mycobacterium species (Mycobacterium tuberculosis complex, MTC).

The probe having the nucleotide sequence of SEQ ID NO: 21 or 60 is specific for the product obtained by carrying out a polymerase chain reaction in the presence of a primer set specific for the IS6110 gene of Mycobacterium tuberculosis which comprises a forward primer having the nucleotide sequence of SEQ ID NO: 58, and a reverse primer having the nucleotide sequence of SEQ ID NO: 59.

In one embodiment of the present invention, the probe for detecting the IS6110 gene of Mycobacterium tuberculosis, having the nucleotide sequence of SEQ ID NO: 21 or 60, may be a Taqman probe.

The nucleotide sequence of SEQ ID NO: 61 can be used as a forward primer specific for the 16S rRNA gene of nontuberculous mycobacteria. The respective nucleotide sequences of SEQ ID NOS: 5 and 8 (NTM-1 and NTM-2) can be used as reverse primers specific for the 16S rRNA gene of nontuberculous mycobacteria.

The probe having the nucleotide sequence of SEQ ID NO: 62 or 63 is specific for the product obtained by carrying out a polymerase chain reaction in the presence of a primer set specific for the 16S rRNA gene of nontuberculous mycobacteria which comprises a forward primer having the nucleotide sequence of SEQ ID NO: 61, and a reverse primer composed of respective primers having the nucleotide sequences of SEQ ID NOS: 5 and 8.

In one embodiment of the present invention, the respective probes for detecting the 16S rRNA of nontuberculous mycobacteria, having the nucleotide sequence of SEQ ID NO: 62 or 63, may be a Taqman probe.

In another embodiment of the present invention, the probe for detecting the IS6110 gene of Mycobacterium tuberculosis is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ. The probe for detecting the 16S rRNA gene of nontuberculous mycobacteria is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1, 2, 3 and MGBNFQ. The fluorescent markers labeled at the 5′ ends may differ from the probe for detecting the IS61110 gene of Mycobacterium tuberculosis to the probe for detecting the 16S rRNA of nontuberculous mycobacteria.

In accordance with yet additional aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59, a probe for detecting the IS6110 gene of Mycobacterium tuberculosis, having the nucleotide sequence of SEQ ID NO: 21 or SEQ ID NO: 60, a primer set specific for the 16S rRNA gene of nontuberculous mycobacteria, comprising a forward primer having the nucleotide sequence of SEQ ID NO: 61, and a reverse primer composed of a primer having the nucleotide sequence of SEQ ID NO: 5 and a primer having the nucleotide sequence of SEQ ID NO: 8; and a probe for detecting the 16S rRNA gene of nontuberculous mycobacteria, having the nucleotide sequence of SEQ ID NO: 62 or 63; and analyzing products of the duplex real-time PCR.

In accordance with yet another additional aspect thereof, the present invention addresses a primer set specific for the 16S rRNA gene of nontuberculous mycobacteria, comprising: a forward primer comprising a primer having the nucleic acid sequence of SEQ ID NO: 65, a primer having the nucleotide sequence of SEQ ID NO: 66, and a primer having the nucleotide sequence of SEQ ID NO: 67; and a reverse primer comprising the nucleotide sequence of SEQ ID NO: 36.

The respective nucleotide sequences of SEQ ID NOS: 65, 66 and 67 (NTM-1, NTM-2, and NTM-3) can be used together as forward primers specific for the 16S rRNA gene of nontuberculous mycobacteria. The forward primer specific for the 16S rRNA gene of nontuberculous mycobacteria is designed to detect all of various nontuberculous mycobacterium species. The nucleotide sequence of SEQ ID NO: 65 (5′-tktggtggaaagcttttgc-3′) is a primer set comprising 5′-tgtggtggaaagcttttgc-3′ (SEQ ID NO: 69) and 5′-tttggtggaaagcttttgc-3′ (SEQ ID NO: 70). In this primer set, for example, SEQ ID NO: 69 and SEQ ID NO: 70 may be present at a ratio of approximately 1:1. The nucleotide sequence of SEQ ID NO: 66 (5′-ggtgwgtggtgcaaagctt-3′) may be a primer set which comprises 5′-ggtgagtggtgcaaagctt-3′ (SEQ ID NO: 71) and 5′-ggtgtgtggtgcaaagctt-3′ (SEQ ID NO: 72) at a ratio of approximately 1:1.

In accordance with a yet further additional aspect thereof, the present invention addresses a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 20; a probe for detecting the IS6110 gene of Mycobacterium tuberculosis, having the nucleotide sequence of SEQ ID NO: 21 or SEQ ID NO: 64; a primer set specific for the 16S rRNA gene of nontuberculous mycobacteria, composed of a forward primer comprising a primer having the nucleotide sequence of SEQ ID NO: 65, a primer having the nucleotide sequence of SEQ ID NO: 66, and a primer having the nucleotide sequence of SEQ ID NO: 67, and a reverse primer having the nucleotide sequence of SEQ ID NO: 36; and a probe for detecting the 16S rRNA gene of nontuberculous mycobacteria, having the nucleotide sequence of SEQ ID NO: 39 or 68.

The detection kit may further comprise a reagent necessary for amplifying DNA by PCR. This reagent may include DNA polymerase, dNTPs, PCR buffer, etc.

According to one embodiment of the present invention, the probe for detecting the IS6110 gene of Mycobacterium tuberculosis, having the nucleotide sequence of SEQ ID NO: 21 or 64, and the probe for detecting the 16S rRNA gene of nontuberculous mycobacteria, having the nucleotide sequence of SEQ ID NO: 39 or 68, may be labeled with different detectable means.

In the kit for the detection of Mycobacterium tuberculosis and nontuberculous mycobacteria, as described above, a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 20 is employed. The primer set specific for the IS6100 gene of Mycobacterium tuberculosis is designed to detect all of various mycobacterium species (Mycobacterium tuberculosis complex, MTC).

The probe having the nucleotide sequence of SEQ ID NO: 21 or 64 is specific for the product obtained by carrying out a polymerase chain reaction in the presence of a primer set specific for the IS6110 gene of Mycobacterium tuberculosis which comprises a forward primer having the nucleotide sequence of SEQ ID NO: 58, and a reverse primer having the nucleotide sequence of SEQ ID NO: 20.

In one embodiment of the present invention, the probe for detecting the IS6110 gene of Mycobacterium tuberculosis, having the nucleotide sequence of SEQ ID NO: 21 or 64, may be a Taqman probe.

The respective sequences of SEQ ID NOS: 65, 66 and 67 may be used as forward primers specific for the 16S rRNA gene of nontuberculous mycobacteria while the nucleotide sequence of SEQ ID NO: 36 may be used as a reverse primer specific for the 16S rRNA of nontuberculous mycobacteria.

The probe having the nucleotide sequence of SEQ ID NO: 39 or 68 is specific for the product obtained by carrying out a polymerase chain reaction in the presence of a primer set specific for the 16S rRNA gene of nontuberculous mycobacteria which comprises a forward primer composed of respective primers having the nucleotide sequences of SEQ ID NOS: 65 to 67, and a reverse primer having the nucleotide sequence of SEQ ID NO: 36.

In one embodiment of the present invention, the probe for detecting the 16S rRNA of nontuberculous mycobacteria, having the nucleotide sequence of SEQ ID NO: 39 and 68, may be a Taqman probe.

The probe having the nucleotide sequence of SEQ ID NO: 68 (5′-FAM-cctgagagggtgwccg-MGB-3′) may be a primer set consisting of 5′-FAM-cctgagagggtgaccg-MGB-3′ (SEQ ID NO: 73) and 5′-FAM-cctgagagggtgtccg-MGB-3′ (SEQ ID NO: 74) at a ratio of 1:1.

In another embodiment of the present invention, the probe for detecting the IS6110 gene of Mycobacterium tuberculosis is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ. The probe for detecting the 16S rRNA gene of nontuberculous mycobacteria is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ. The fluorescent markers labeled at the 5′ ends may differ from the probe for detecting the IS61110 gene of Mycobacterium tuberculosis to the probe for detecting the 16S rRNA of nontuberculous mycobacteria.

In accordance with a still yet additional aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 20, a probe for detecting the IS6110 gene of Mycobacterium tuberculosis, having the nucleotide sequence of SEQ ID NO: 21 or SEQ ID NO: 64, a primer set specific for the 16S rRNA gene of nontuberculous mycobacteria, composed of a forward primer comprising a primer having the nucleotide sequence of SEQ ID NO: 65, a primer having the nucleotide sequence of SEQ ID NO: 66, and a primer having the nucleotide sequence of SEQ ID NO: 67, and a reverse primer having the nucleotide sequence of SEQ ID NO: 36, and a probe for detecting the 16S rRNA gene of nontuberculous mycobacteria, having the nucleotide sequence of SEQ ID NO: 39 or 68; and analyzing products of the duplex real-time PCR.

In accordance with still yet another additional aspect thereof, the present invention addresses a kit for the detection of Mycobacterium tuberculosis and nontuberculous mycobacteria, comprising: a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59; a probe for detecting the IS6110 gene of Mycobacterium tuberculosis, having the nucleotide sequence of SEQ ID NO: 60; a primer set specific for the 16S rRNA gene of nontuberculous mycobacteria, composed of a forward primer having the nucleotide sequence of SEQ ID NO: 24, and a reverse primer having the nucleotide sequence of SEQ ID NO: 75; and a probe for detecting the 16S rRNA gene of nontuberculous mycobacteria, comprising a probe having the nucleotide sequence of SEQ ID NO: 27 and a probe having the nucleotide sequence of SEQ ID NO: 76.

The detection kit may further comprise a reagent necessary for amplifying DNA by PCR. This reagent may include DNA polymerase, dNTPs, PCR buffer, etc.

According to one embodiment of the present invention, the probe for detecting the IS6110 gene of Mycobacterium tuberculosis, having the nucleotide sequence of SEQ ID NO: 60, and the probe for detecting the 16S rRNA gene of nontuberculous mycobacteria, composed of the respective probes NTM-1 and NTM-2 having the nucleotide sequences of SEQ ID NOS: 27 and 76, may be labeled with different detectable means.

In the kit for the detection of Mycobacterium tuberculosis and nontuberculous mycobacteria, as described above, a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59 is employed. The primer set specific for the IS6100 gene of Mycobacterium tuberculosis is designed to detect all of various mycobacterium species (Mycobacterium tuberculosis complex, MTC).

For use in the detection of the IS6110 gene of Mycobacterium tuberculosis, the probe having the nucleotide sequence of SEQ ID NO: 60 is specific for the product obtained by carrying out a polymerase chain reaction in the presence of a primer set specific for the IS6110 gene of Mycobacterium tuberculosis which comprises a forward primer having the nucleotide sequence of SEQ ID NO: 58, and a reverse primer having the nucleotide sequence of SEQ ID NO: 59.

According to one embodiment of the present invention, the probe for detecting the IS6110 gene of Mycobacterium tuberculosis, having the nucleotide sequence of SEQ ID NO: 60, may be a Taqman probe.

In the kit for the detection of Mycobacterium tuberculosis and nontuberculous mycobacteria, as described above, a primer set specific for the 16S rRNA gene of nontuberculous mycobacteria, composed of a forward primer having the nucleotide sequence of SEQ ID NO: 24, and a reverse primer having the nucleotide sequence of SEQ ID NO: 75 is also employed. The forward primer having the nucleotide sequence of SEQ ID NO: 24, and the reverse primer having the nucleotide sequence of SEQ ID NO: 75 form a primer set, together, which is universally used to amplify the 16S rRNA gene of both MTC and NTM.

The nucleotide sequence of SEQ ID NO: 24 (5′-ggataagcytgggaaactgg-3′) may serve as a forward primer specific for the 16S rRNA gene of mycobacteria, and may be a primer set comprising 5′-ggataagcctgggaaactgg-3′ (SEQ ID NO: 29) and 5′-ggataagcttgggaaactgg-3′ (SEQ ID NO: 30) at a ratio of approximately 1:1.

The nucleotide sequence of SEQ ID NO: 36 may be a reverse primer specific for the 16S rRNA of mycobacteria.

For use in the detection of the 16S rRNA gene of nontuberculous mycobacteria, the probe comprising the respective probes having the nucleotide sequences of SEQ ID NOS: 27 and 76 is specific for the product obtained by carrying out a polymerase chain reaction in the presence of a primer set specific for the 16s rRNA gene of nontuberculous mycobacteria which comprises a forward primer having the nucleotide sequence of SEQ ID NO: 24, and a reverse primer having the nucleotide sequence of SEQ ID NO: 75.

The probe for detecting the 16S rRNA gene of nontuberculous mycobacteria, comprising probes having the nucleotide sequences of SEQ ID NOS: 27 and 76, is designed to detect all of various nontuberculous mycobacterium species.

In another embodiment of the present invention, the probe for detecting the IS6110 gene of Mycobacterium tuberculosis is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ. The probe for detecting the 16S rRNA gene of nontuberculous mycobacteria is labeled at the 5′ end with a fluorescent marker selected from the group consisting of FAM, VIC, TET, JOE, HEX, CY3, CY5, ROX, RED610, TEXAS RED, RED670, and NED, and at the 3′ end with a fluorescence quencher selected from the group consisting of 6-TAMRA, BHQ-1,2,3 and MGBNFQ. The fluorescent markers labeled at the 5′ ends may differ from the probe for detecting the IS61110 gene of Mycobacterium tuberculosis to the probe for detecting the 16S rRNA of nontuberculous mycobacteria.

In accordance with a still yet further additional aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, consisting of a forward primer having the nucleotide sequence of SEQ ID NO: 58 and a reverse primer having the nucleotide sequence of SEQ ID NO: 59, a probe for detecting the IS6110 gene of Mycobacterium tuberculosis, having the nucleotide sequence of SEQ ID NO: 60, a primer set specific for the 16S rRNA gene of nontuberculous mycobacteria, composed of a forward primer having the nucleotide sequence of SEQ ID NO: 24, and a reverse primer having the nucleotide sequence of SEQ ID NO: 75, and a probe for detecting the 16S rRNA gene of nontuberculous mycobacteria, comprising a probe having the nucleotide sequence of SEQ ID NO: 27 and a probe having the nucleotide sequence of SEQ ID NO: 76; and analyzing products of the duplex real-time PCR.

### MODE FOR INVENTION

A better understanding of the present invention may be obtained through the following examples which are set forth to illustrate, but are not to be construed as limiting the present invention.

### REFERENCE EXAMPLE

Search For Nucleotide Sequence Characteristic of Mycobacterium tuberculosis and Nontuberculous Mycobacteria

1. Target and Gene Loci

Used in searching for nucleotide sequences characteristic of Mycobacterium tuberculosis and nontuberculous mycobacteria were data of 16S ribosomal RNA genes of the following mycobacteria:

M. abscessus (AJ419970.1, AJ416940.1, AJ536038), M. acapulcensis (AF480575.1), M. africanum (AF480605.1), M. agri (AJ429045.1), M. aichiense (X55598.1), M. alvei (NR_{—}024859.1), M. asiaticum (X55604.1), M. aurum (FJ172298.1), M. austroafricanum (GU121552.1), M. avium (NR_{—}025584.1, AJ536037.1, EF521892.1), M. bohemicum (NR_{—}026054.1), M. botniense (NR_{—}028878.1), M. bovis (GU142937.1), M. branderi (AF480574.1), M. brumae (NR_{—}025233.1), M. celatum (L08169.1), M. chelonae (AM884324.1, AJ419969.1), M. chitae (NR_{—}029220.1), M. chlorophenolicum (NR_{—}026173.1), M. chubuense (X55596.1), M. confluentis (AJ634379.1), M. conspicuum (X029298.1), M. cookii (X53896.1), M. diernhoferi (AF480599.1), M. doricum (NR_{—}025099.1), M. duvalii (NR_{—}026073.1), M. engbaekii (AF480577.1), M. fallax (AF480600.1), M. farcinogenes (X55592.1), M. flavescens (AY734993.1), M. fortuitum (AY457066.1, AF480580.1, GU142933.1), M. gadium (NR_{—}026087.1), M. gastri (GU142918.1), M. genavense (NR_{—}029223.1), M. gilvum (AB491971.1), M. goodii (AY457079.1), M. gordonae (GU142923.1), M. haemophilum (V06638.1), M. hassiacum (NR_{—}026011.1), M. heidelbergense (NR_{—}025268.1), M. hiberniae (NR_{—}026092.1), M. hodleri (NR_{—}026286.1), M. immunogen (AJ011771.1), M. interjectum (X70961.1), M. intermedium (X67847.1), M. intracellulare (AY652958.1, AJ536036.1, X52927.1, M61684.1), M. kansasii (M29575.1, X15916.1), M. lentiflavum (AF480583.1), M. mageritense (AY457076.1), M. malmoense (GQ153278.1), M. marinum (AF456238.1, AY513243.1), M. microti (NR_{—}025234.1), M. monacense (GU142931.1), M. moriokaense (AY859686.1), M. mucogenicum (AF480585.1), M. neoaurum (FJ172306.1), M. nonchromogenicum (DQ058406.1), M. obuense (X55597.1), M. paraffinicum (GQ153282.1), M. parafortuitum (NR_{—}026285.1), M. peregrinum (AY457069.1), M. phlei (AF480603.1), M. porcinum (AY457077.1), M. poriferae (NR_{—}025235.1), M. pulveris (NR_{—}025528.1), M. rhodesiae (NR_{—}025529.1), M. scrofulaceum (GQ153271.1), M. senuense (DQ536409.1), M. septicum (AY457070.1), M. shimoidei (X82459.1), M. simiae (GQ153280.1), M. smegmatis (NR_{—}025311.1), M. sphagni (X55590.1), M. szulgai (X52926.1), M. terrae (NR_{—}029168.1), M. thermoresistibile (GU142928.1), M. tilburgii (AJ580826.1), M. triplex (GQ153279.1), M. triviale (DQ058405.1), M. tuberculosis (GU142936.1, GU142935.1, AY53603.1, X55588.1, X52917.1), M. tusciae (NR_{—}024903.1), M. ulcerans (Z13990.1), M. vaccae (X55601.1), M. wolinskyi (AY457083.1), M. xenopi (X52929.1). The data of the 16S ribosomal RNA genes were obtained from the database of the NCBI (National center for Biotechnology Information).

Analysis of the data of 16S rRNA gene sequences of the mycobacterium species with the aid of Sequencher 4.9 resulted in characteristic nucleotide sequences, that is, nucleotides characteristic of Mycobacterium tuberculosis complex, and nucleotides absent from Mycobacterium tuberculosis complex, but intrinsic to nontuberculous mycobacteria.

### Example 1

### Separation and Detection of Mycobacterium tuberculosis Complex and Nontuberculous Mycobacteria 1

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). The Taqman probes and the primers used in the detection of the target genes were designed using the Primer3 program.

(1) MTC

1) target gene: IS6110

2) primers

a. forward primer:
(SEQ ID NO: 1)
5′-cgaactcaaggagcacatca-3′
b. reverse primer:
(SEQ ID NO: 2)
5′-agtttggtcatcagccgttc-3′

3) Taqman probe

(SEQ ID NO: 3)
5′-VIC-agtgtggctaaccctgaa-MGB-3′

4) PCR product size: 135 bp

(2) NTM

1) target gene: 16S rRNA

2) primers

a. forward primer:
(SEQ ID NO: 4)
5′-ggyrayctgccctgcac-3′
b. reverse primers
NTM-1:
(SEQ ID NO: 5)
5′-cccacaccgcaaaagctt-3′,
(SEQ ID NO: 6)
5′-cccacaccgcaaaagct-3′,
or
(SEQ ID NO: 7)
5′-tcccacaccgcaaaagct-3′
NTM-2:
(SEQ ID NO: 8)
5′-catcccacaccgctaccw-3′

3) Taqman probe

(SEQ ID NO: 9)
5′-FAM-cggtattagacccagtttcc-MGB-3′

4) PCR product size: 104 bp

### Example 1-1

### Duplex Real-Time PCR Using the Nucleotide Sequence of SEQ ID NO: 5 as a Reverse Primer NTM-1 for the Detection of NTM

(1) Isolation of DNA

DNA was isolated from 186 Mycobacterium species and 78 nontuberculous mycobacterium species, all recovered from clinical specimens, and from 7 standard ATCC mycobacteria species including M. tuberculosis (ATCC 25177), M. intracellulare (ATCC 13950), M. scrofulaceum (ATCC 19981), M. kansasii (ATCC 12478), M. fortuitum (ATCC 6841), M. abscessus (ATCC 19977), and M. avium (ATCC 25291).

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC standard species were cultured in broths.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 15 sec, and annealing and elongation at 66° C. for 15 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 1, below. In the primer-probes Mix, a forward primer and a reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume as in MTC. The nucleotide sequences of SEQ ID NOS: 5 and 6 were used as NTM-1 and NTM-2 reverse primers, respectively. For the NTM-2 reverse primer, 5′-catcccacaccgctacct-3′ (SEQ ID NO: 10) and 5′-catcccacaccgctacca-3′ (SEQ ID NO: 11) were used in the same amounts. An NTM forward primer was a set of 5′-ggtaatctgccctgcac-3′ (SEQ ID NO: 12), 5′-ggtaacctgccctgcac-3′ (SEQ ID NO: 13), 5′-ggcaatctgccctgcac-3′ (SEQ ID NO: 14), 5′-ggcaacctgccctgcac-3′ (SEQ ID NO: 15), 5′-ggtgatctgccctgcac-3′ (SEQ ID NO: 16), 5′-ggtgacctgccctgcac-3′ (SEQ ID NO: 17), 5′-ggcgatctgccctgcac-3′ (SEQ ID NO: 18), and 5′-ggcgacctgccctgcac-3′ (SEQ ID NO: 19). In the primer set, the primers of SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19 were used at a ratio of about 1:1:1:1:1:1:1:1.

TABLE 1

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™ and VIC™ were monitored in a green channel (510±5 nm) and a yellow channel (555±5 nm), respectively.

### Example 1-2

### Duplex Real-Time PCR Using the Nucleotide Sequence of SEQ ID NO: 6 as the Reverse Primer NTM-1 for the Detection of NTM

Duplex real-time PCR was carried out in the same manner as in Example 1-1, with the exception that the nucleotide sequence of SEQ ID NO: 6 was used as the reverse primer NTM-1.

### Example 1-3

### Duplex Real-Time PCR Using the Nucleotide Sequence of SEQ ID NO: 7 as the Reverse Primer NTM-1 for the Detection of NTM

Duplex real-time PCR was carried out in the same manner as in Example 1-1, with the exception that the nucleotide sequence of SEQ ID NO: 6 was used as the reverse primer NTM-1.

2. Result of Duplex Real-Time PCR

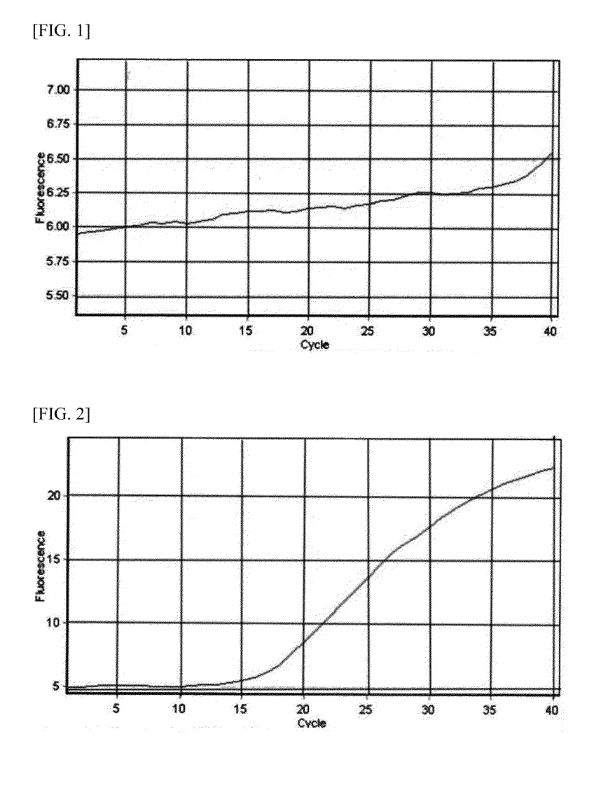

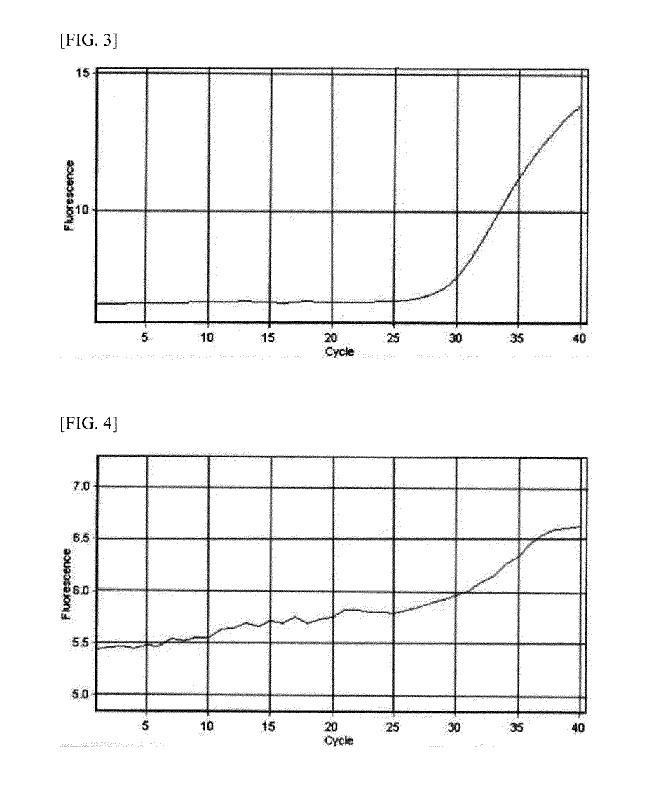

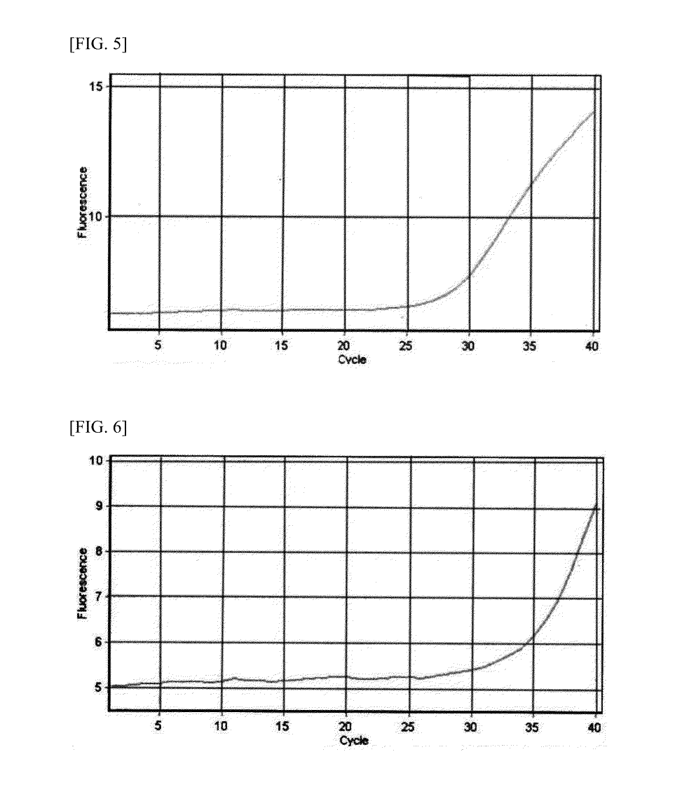

FIGS. 1 to 6 show results of the duplex real-time PCR performed with the mycobacteria. In the graphs, the number of cycles of PCR is set on the X-axis while fluorescence intensity (F) is on the Y-axis. FIGS. 1 and 2 are graphs in which fluorescence intensities in green and yellow channels are plotted, respectively, against the number of cycles of real-time PCR with MTC. FIGS. 3 and 4 are graphs in which fluorescence intensities in green and yellow channels are plotted, respectively, against the number of cycles of real-time PCR with NTM. FIGS. 5 and 6 are graphs in which fluorescence intensities in green and yellow channels are plotted, respectively, against the number of cycles of real-time PCR with MTC+NTM.

As can be seen in FIGS. 1 to 6, the IS6110 gene amplified from MTC was detected in the yellow channel and the 16S rRNA gene amplified from NTM in the green channel. Therefore, the duplex real-time PCR using the primers and probes according to the present invention was found to guarantee the detection of MTC and NTM, simultaneously, with high reliability.

### Example 2

### Separation and Detection of Mycobacterium tuberculosis Complex and Nontuberculous Mycobacteria 2

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). The Taqman probes and the primers used in the detection of the target genes were designed using the Primer3 program.

(1) MTC

1) target gene: IS6110

2) primers

a. forward primer:
(SEQ ID NO: 1)
5′-cgaactcaaggagcacatca-3′
b. reverse primer:
(SEQ ID NO: 20)
5′-cagggttagccacactttgc-3′

3) Taqman probe

(SEQ ID NO: 21)
5′-HEX-cgccaactacggtgtttacggtg-BHQ1-3′

4) PCR product size: 79 bp

(2) NTM

1) target gene: 16S rRNA

2) primers

a. forward primer:
(SEQ ID NO: 22)
5′-gtggcgaacgggtgagtaa-3′
b. reverse primer
NTM-1:
(SEQ ID NO: 5)
5′-cccacaccgcaaaagctt-3′,
(SEQ ID NO: 6)
5′-cccacaccgcaaaagct-3′,
or
(SEQ ID NO: 7)
5′-tcccacaccgcaaaagct-3′
NTM-2:
(SEQ ID NO: 8)
5′-catcccacaccgctaccw-3′

3) Taqman probe

(SEQ ID NO: 23)
5′-FAM-cggtattagacccagtttcccaggct-BHQ1-3′

4) PCR product size: 128 bp

### Example 2-1

Duplex Real-Time PCR Using the Nucleotide Sequence of SEQ ID NO: 5 as a Reverse Primer NTM-1 for the Detection of NTM

(1) Isolation of DNA

DNA was isolated from 186 Mycobacterium species and 78 nontuberculous mycobacterium species, all recovered from clinical specimens, and from 7 standard ATCC mycobacteria species including M. tuberculosis (ATCC 25177), M. intracellulare (ATCC 13950), M. scrofulaceum (ATCC 19981), M. kansasii (ATCC 12478), M. fortuitum (ATCC 6841), M. abscessus (ATCC 19977), and M. avium (ATCC 25291).

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC standard species were cultured in broths.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 10 sec, and annealing and elongation at 65° C. for 15 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 2, below. In the primer-probes Mix, a forward primer and a reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume as in MTC. The nucleotide sequences of SEQ ID NOS: 5 and 8 were used as NTM-1 and NTM-2 reverse primers, respectively. For the NTM-2 reverse primer, 5′-catcccacaccgctacct-3′ (SEQ ID NO: 10) and 5′-catcccacaccgctacca-3′ (SEQ ID NO: 11) were used in the same amounts.

TABLE 2

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™ and VIC™ were monitored in a green channel (510±5 nm) and a yellow channel (555±5 nm), respectively.

### Example 2-2

### Duplex Real-Time PCR Using the Nucleotide Sequence of SEQ ID NO: 6 as the Reverse Primer NTM-1 for the Detection of NTM

Duplex real-time PCR was carried out in the same manner as in Example 2-1, with the exception that the nucleotide sequence of SEQ ID NO: 6 was used as the reverse primer NTM-1.

### Example 2-3

### Duplex Real-Time PCR Using the Nucleotide Sequence of SEQ ID NO: 7 as the Reverse Primer NTM-1 for the Detection of NTM

Duplex real-time PCR was carried out in the same manner as in Example 2-1, with the exception that the nucleotide sequence of SEQ ID NO: 7 was used as the reverse primer NTM-1.

2. Result of Duplex Real-Time PCR

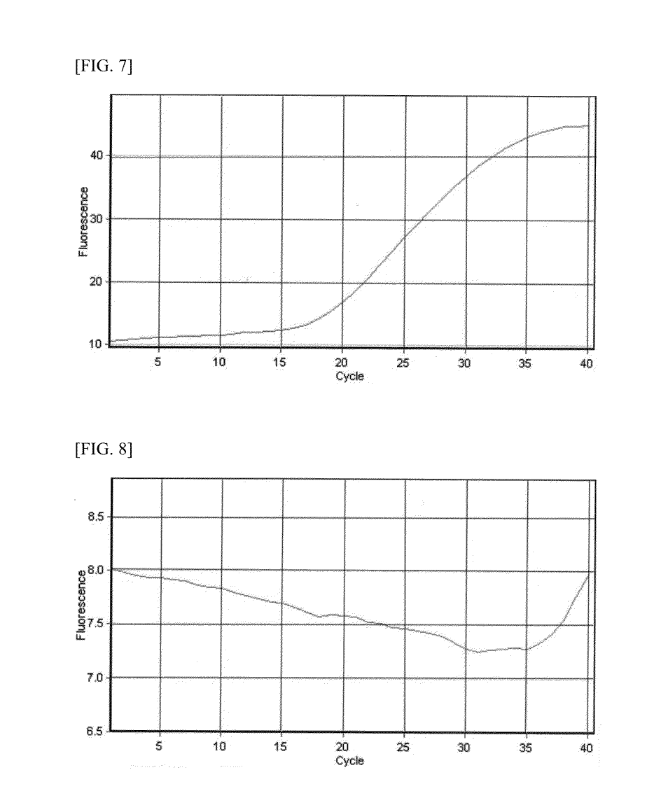

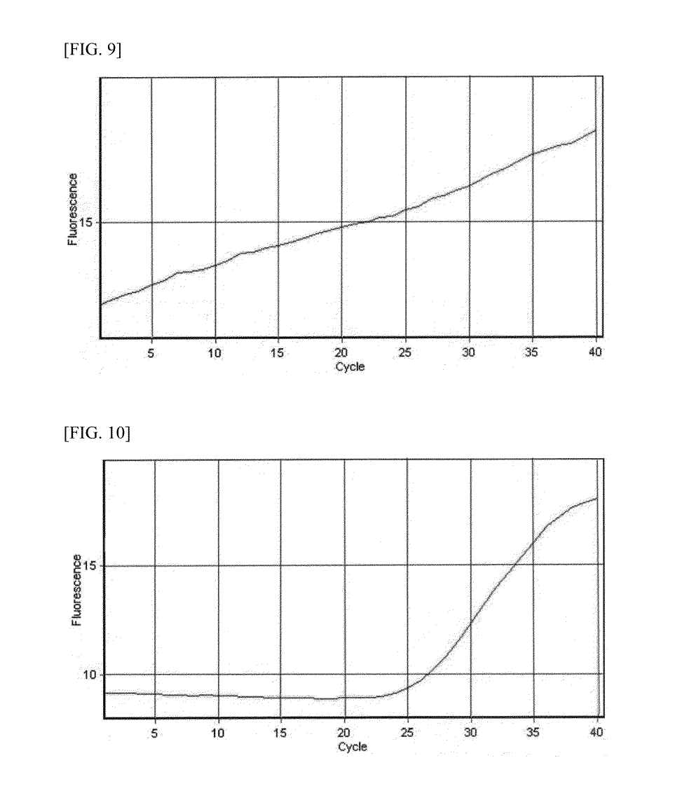

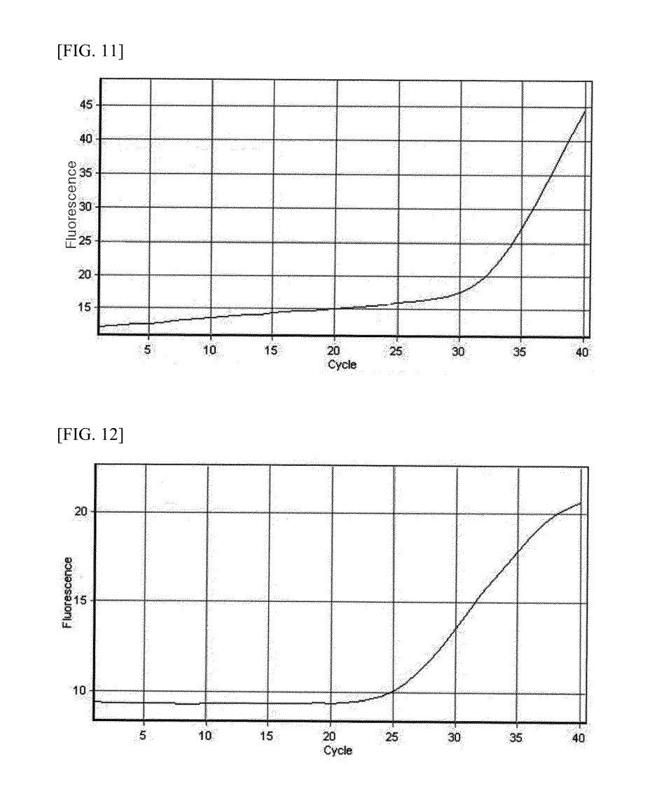

FIGS. 7 to 12 show results of the duplex real-time PCR performed with the mycobacteria. In the graphs, the number of cycles of PCR is set on the X-axis while fluorescence intensity (F) is on the Y-axis. FIGS. 7 and 8 are graphs in which fluorescence intensities in yellow and green channels are plotted, respectively, against the number of cycles of real-time PCR with MTC. FIGS. 9 and 10 are graphs in which fluorescence intensities in yellow and green channels are plotted, respectively, against the number of cycles of real-time PCR with NTM. FIGS. 11 and 12 are graphs in which fluorescence intensities in yellow and green channels are plotted, respectively, against the number of cycles of real-time PCR with MTC+NTM.

As can be seen in FIGS. 7 to 12, the IS6110 gene amplified from MTC was detected in the yellow channel and the 16S rRNA gene amplified from NTM in the green channel. Therefore, the duplex real-time PCR using the primers and the probe according to the present invention was found to guarantee the detection of MTC and NTM, simultaneously, with high reliability.

### Example 3

### Separation and Detection of Mycobacterium tuberculosis Complex and Nontuberculous Mycobacteria 3

1. Detection Target and Primer Design

A universal primer was used to amplify the 16S rRNA gene of mycobacteria. Nucleotide sequences of 16S rRNA genes characteristic of MTC (M. tuberculosis, M. bovis, M. africanum, M. microti), and NTM were used as Taqman probes. These probes were designed using the Primer3 program.

(1) universal primers (target gene: 16S rRNA)

a. forward primer:
(SEQ ID NO: 24)
5′-ggataagcytgggaaactgg-3′
b. reverse primer:
(SEQ ID NO: 25)
5′-accccaccaacwagctgata-3′

(2) Taqman probe (target gene: 16S rRNA)

a. MTC Taqman probe
(SEQ ID NO: 26)
5′-VIC-tggtggaaagcgcttta-MGB-3′
b. NTM Taqman probe
NTM-1:
(SEQ ID NO: 27)
5′-FAM-tggtggaaagcttttgc-MGB-3′
NTM-2:
(SEQ ID NO: 28)
5′-FAM-tggaaagygtttggtagc-MGB-3′

2. Duplex real-time PCR

(1) Isolation of DNA

DNA was isolated from 186 Mycobacterium species and 78 nontuberculous mycobacterium species, all recovered from clinical specimens, and from 7 standard ATCC mycobacteria species including M. tuberculosis (ATCC 25177), M. intracellulare (ATCC 13950), M. scrofulaceum (ATCC 19981), M. kansasii (ATCC 12478), M. fortuitum (ATCC 6841), M. abscessus (ATCC 19977), and M. avium (ATCC 25291).

The species identified in clinical subjects including 186 Mycobacerium species and 78 nontuberculous mycobacterium species were either detected in a liquid medium (MGIT mycobacterium medium) or a solid medium (Ogawa medium) or isolated directly from sputum specimens. The ATCC standard species were cultured in broths.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 15 sec, and annealing and elongation at 66° C., using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 3, below. In the primer-probes Mix, the universal primer for mycobacteria comprised a forward primer and a reverse primer in the same amounts (10 pmoles/μL), with each of MTC, NTM-1 and NTM-2 probes present in an amount of 4 pmoles/μL. Accordingly, 1.25 μL of the primer-probes mix for MTC contained the forward primer and the reverse primer in an amount of 12.5 pmoles, each, and the MTC, NTM-1, and NTM-2 probes in an amount of 5 pmoles, each. Since a total volume of PCR mixture was 25 μL, it contained the primers at a concentration of 0.5 μM (12.5 pmoles/25 μL), and each of the MTC, NTM-1, NTM-2 probes at a concentration of 0.2 μM (5 pmoles/25 μL). In this regard, the forward primer having the nucleotide sequence of SEQ ID NO: 24 was designed to contain 5′-ggataagcctgggaaactgg-3′ (SEQ ID NO: 29) and 5′-ggataagcttgggaaactgg-3′ (SEQ ID NO: 30) in an amount of 6.25 pmoles, each. As for the reverse primer having the nucleotide sequence of SEQ ID NO: 25, it contained 5′-accccaccaacaagctgata-3′ (SEQ ID NO: 31) and 5′-accccaccaactagctgata-3′ (SEQ ID NO: 32) in an amount of 6.25 pmoles, each. In the NTM-2 probe having the nucleotide sequence of SEQ ID NO: 28, 5′-FAM-tggaaagcgtttggtagc-MGB-3′(SEQ ID NO: 33) and 5′-FAM-tggaaagtgtttggtagc-MGB-3′ (SEQ ID NO: 34) were contained in an amount of 2.5 pmoles, each.

TABLE 3

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™ and VIC™ were monitored in a green channel (510±5 nm) and a yellow channel (555±5 nm), respectively.

3. Result of Duplex Real-Time PCR

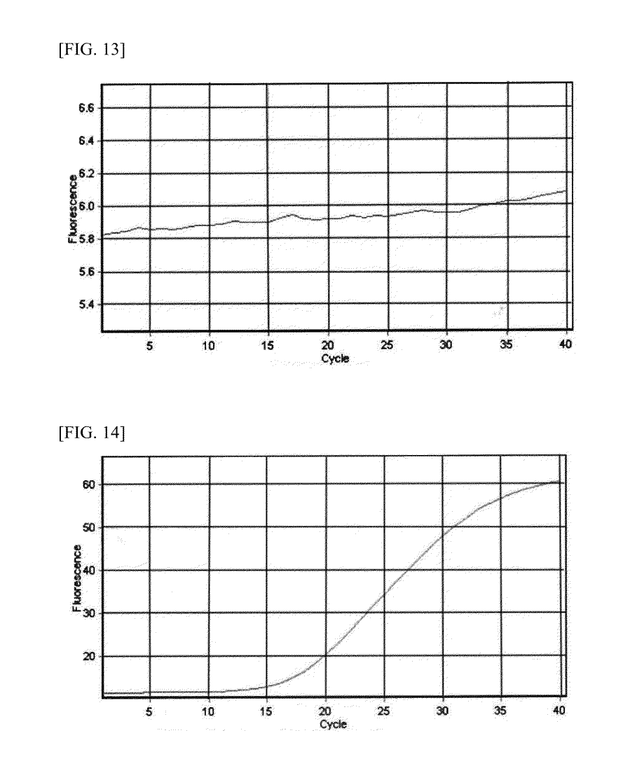

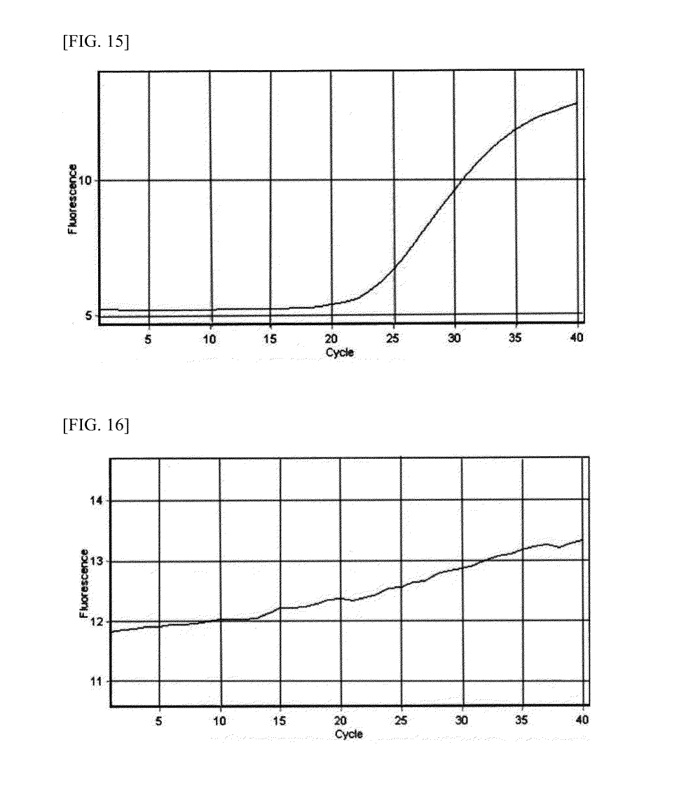

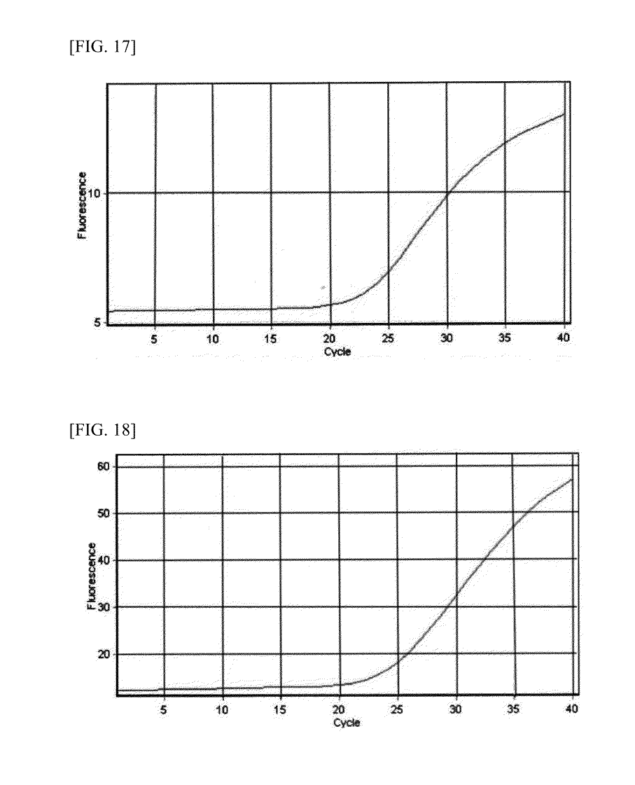

FIGS. 13 to 18 show results of the duplex real-time PCR performed with the mycobacteria. In the graphs, the number of cycles of PCR is set on the X-axis while fluorescence intensity (F) is on the Y-axis. FIGS. 13 and 14 are graphs in which fluorescence intensities in green and yellow channels are plotted, respectively, against the number of cycles of real-time PCR with MTC. FIGS. 15 and 16 are graphs in which fluorescence intensities in green and yellow channels are plotted, respectively, against the number of cycles of real-time PCR with NTM. FIGS. 17 and 18 are graphs in which fluorescence intensities in green and yellow channels are plotted, respectively, against the number of cycles of real-time PCR with MTC+NTM.

As can be seen in FIGS. 13 to 18, the IS6110 gene amplified from MTC was detected in the yellow channel and the 16S rRNA gene amplified from NTM in the green channel. Therefore, the duplex real-time PCR using the primers and the probes according to the present invention was found to guarantee the detection of MTC and NTM, simultaneously, with high reliability.

### Example 4

### Separation and Detection of Mycobacterium tuberculosis Complex and Nontuberculous Mycobacteria 4

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). The Taqman probes and the primers used in the detection of the target genes were designed using the Primer3 program.

(1) MTC

1) target gene: IS6110

2) primer

a. forward primer:
(SEQ ID NO: 1)
5′-cgaactcaaggagcacatca-3′
b. reverse primer:
(SEQ ID NO: 20)
5′-cagggttagccacactttgc-3′

3) Taqman probe

(SEQ ID NO: 21)
5′-HEX-cgccaactacggtgtttacggtg-BHQ1-3′

4) PCR product size: 79 bp

(2) NTM

1) target gene: 16S rRNA

2) primer

a. forward primer:
(SEQ ID NO: 35)
5′-catgtyttstggkgsaaagctt-3′
b. reverse primer:
(SEQ ID NO: 36)
5′-cgtaggagtctgggccgta-3′

3) Taqman probe

(SEQ ID NO:37)
FAM-tagccggcctgagagggtg-BHQ1,
(SEQ ID NO: 38)
FAM-cctgagagggtgwccggcc-BHQ1
or
(SEQ ID NO: 39)
FAM-cgggtagccggcctgagag-BHQ1

4) PCR product size: 152 bp

### Example 4-1

Duplex Real-Time PCR Using the Nucleotide Sequence of SEQ ID NO: 37 as a Taqman Probe for the Detection of NTM

(1) Isolation of DNA

DNA was isolated from 186 Mycobacterium species and 78 nontuberculous mycobacterium species, all recovered from clinical specimens, and from 68 standard mycobacteria species including M. abscessus ATCC 19977, M. acapulcensis KCTC 9501, M. africanum ATCC 25420, M. agri KCTC 9502, M. alvei KCTC 19709, M. asiaticum KCTC 9503, M. aurum KCTC 19457, M. austroafricanum KCTC 9504, M. avium ATCC 25291, M. bolletii KCTC 19281, M. botniense KCTC 19646, M. bovis ATCC 19210, M. brumae KCTC 19711, M. celatum ATCC 51131, M. chelonae subsp chelonae KCTC 9505, M. chlorophenolicum KCTC 19089, M. chubuense KCTC 19712, M. diernhoferi KCTC 9506, M. fallax KCTC 9508, M. flavescens ATCC 14474, M. fortuitum ATCC 6841, M. frederiksbergense KCTC 19100, M. gadium ATCC 27726, M. gastri ATCC 15754, M. gilvum KCTC 19423, M. goodii ATCC BAA-955, M. gordonae KCTC 9513, M. haemophilum ATCC 29548, M. hassiacum ATCC 700660, M. interjectum ATCC 51457, M. intermedium ATCC 51848, M. intracellulare ATCC 13950, M. intracellulare KCTC 9514, M. kansasii ATCC 12478, M. lentiflavum KMRC 70087, M. malmoense ATCC 29571, M. mantobense KCTC 9977, M. marinum ATCC 927, M. massiliense KCTC 19086, M. microti ATCC 19422, M. moriokaense KCTC 9516, M. mucogenicum KCTC 19088, M. neoaurum KCTC 19096, M. nonchromogenicum ATCC 19530, M. obuense KCTC 19097, M. parascrofulaceum KCTC 9979, M. peregrinum KCTC 9615, KMRC 75002, M. phlei KCTC 9689, M. porcinum KCTC 9517, M. pulveris KCTC 9518, M. scrofulaceum ATCC 19981, M. septicum ATCC 700731, M. simiae ATCC 25275, M. shimoidei ATCC 27962, M. smegmatis KCTC 9108, M. szulgai KCTC 9520, KMRC 31125, M. terrae KCTC 9614, M. triplex ATCC 700071, M. triviale KMRC 70093, M. tuberculosis ATCC 25177, ATCC 27294, M. ulcerans ATCC 19423, M. vaccae KCTC 19087, M. vanbaalenii KCTC 9966, M. wolinskyi ATCC 700010, and M. xenopi KMRC 42001.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and the KCTC species were cultured in broths while the KMRC species was cultured on an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 12 sec, and annealing and elongation at 63° C. for 12 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 4, below. In the primer-probes Mix, the forward primer and the reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume; as in MTC. The NTM forward primer (SEQ ID NO: 35) was a set of 5′-catgtcttgtgggggaaagctt-3′ (SEQ ID NO: 40), 5′-catgttttgtgggggaaagctt-3′ (SEQ ID NO: 41), 5′-catgtcttctgggggaaagctt-3′ (SEQ ID NO: 42), 5′-catgtcttgtggtggaaagctt-3′ (SEQ ID NO: 43), 5′-catgtcttgtggggcaaagctt-3′ (SEQ ID NO: 44), 5′-catgttttctgggggaaagctt-3′ (SEQ ID NO: 45), 5′-catgtcttctggtggaaagctt-3′ (SEQ ID NO: 46), 5′-catgtcttgtggtgcaaagctt-3′ (SEQ ID NO: 47), 5′-catgttttgtggggcaaagctt-3′ (SEQ ID NO: 48), 5′-catgtcttctggggcaaagctt-3′ (SEQ ID NO: 49), 5′-catgttttgtggtggaaagctt-3′ (SEQ ID NO: 50), 5′-catgttttctggtggaaagctt-3′ (SEQ ID NO: 51), 5′-catgttttctggggcaaagctt-3′ (SEQ ID NO: 52), 5′-catgttttgtggtgcaaagctt-3′ (SEQ ID NO: 53), 5′-catgtcttctggtgcaaagctt-3′ (SEQ ID NO: 54), and 5′-catgttttctggtgcaaagctt-3′ (SEQ ID NO: 55) which were present at a ratio of about 1:1:1:1:1:1:1:1:1:1:1:1:1:1:1:1.

TABLE 4

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™ and HEX™ were monitored in a green channel (510±5 nm) and a yellow channel (555±5 nm), respectively.

### Example 4-2

### Duplex Real-Time PCR Using the Nucleotide Sequence of SEQ ID NO: 38 as a Taqman Probe for the Detection of NTM

Duplex real-time PCR was carried out in the same manner as in Example 4-1, with the exception that the nucleotide sequence of SEQ ID NO: 38 was used as a Taqman probe for the detection of NTM. The probe of SEQ ID NO: 38 was designed to contain FAM-cctgagagggtgaccggcc-BHQ1 (SEQ ID NO: 56) and FAM-cctgagagggtgtccggcc-BHQ1 (SEQ ID NO: 57) in the same amounts.

### Example 4-3

### Duplex Real-Time PCR Using the Nucleotide Sequence of SEQ ID NO: 39 as a Taqman Probe for the Detection of NTM

Duplex real-time PCR was carried out in the same manner as in Example 4-1, with the exception that the nucleotide sequence of SEQ ID NO: 39 was used as a Taqman probe for the detection of NTM.

2. Result of Duplex Real-Time PCR

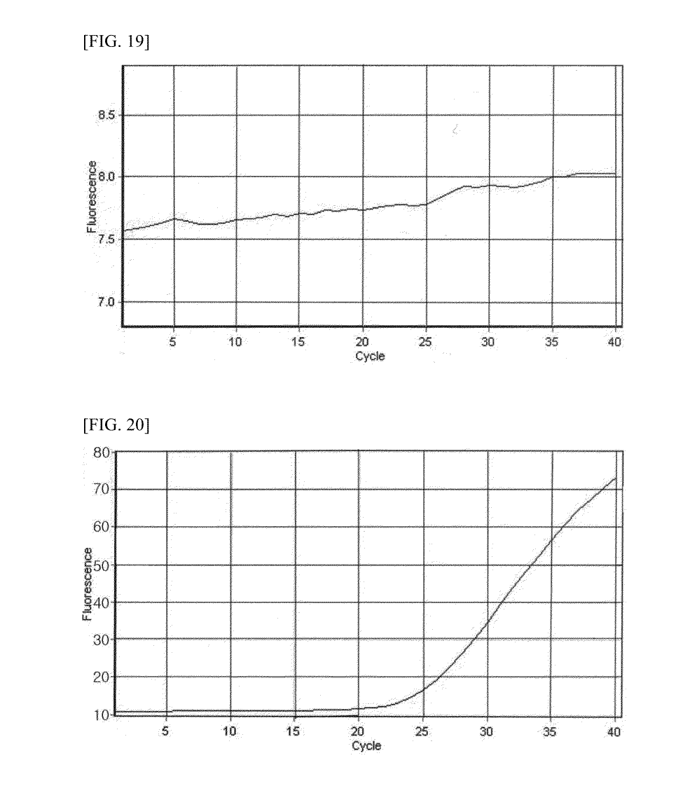

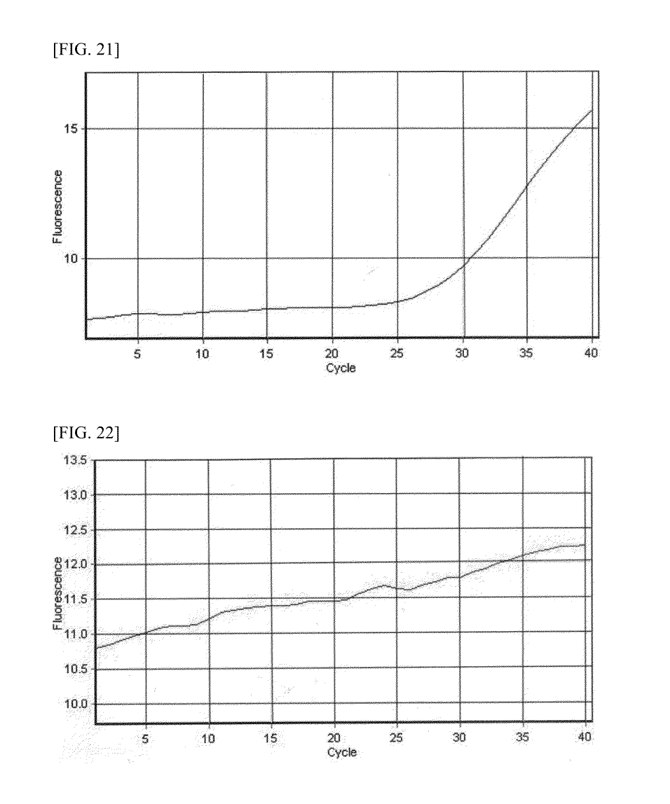

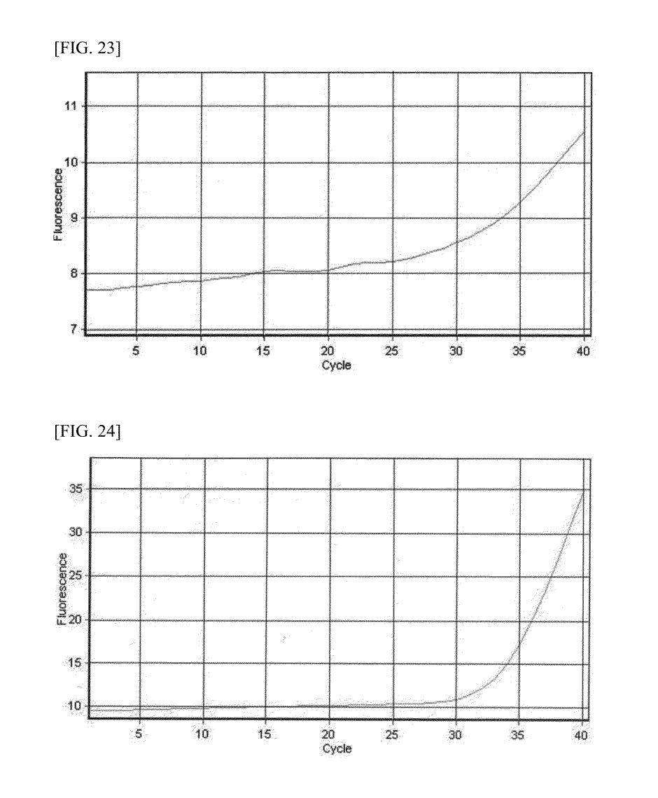

FIGS. 19 to 24 show results of the duplex real-time PCR performed with the mycobacteria. In the graphs, the number of cycles of PCR is set on the X-axis while fluorescence intensity (F) is on the Y-axis. FIGS. 19 and 20 are graphs in which fluorescence intensities in green and yellow channels are plotted, respectively, against the number of cycles of real-time PCR with MTC. FIGS. 21 and 22 are graphs in which fluorescence intensities in green and yellow channels are plotted, respectively, against the number of cycles of real-time PCR with NTM. FIGS. 23 and 24 are graphs in which fluorescence intensities in green and yellow channels are plotted, respectively, against the number of cycles of real-time PCR with MTC+NTM.

As can be seen in FIGS. 19 to 24, the IS6110 gene amplified from MTC was detected in the yellow channel and the 16S rRNA gene amplified from NTM in the green channel. Therefore, the duplex real-time PCR using the primers and the probes according to the present invention was found to guarantee the detection of MTC and NTM, simultaneously, with high reliability.

### Example 5

### Separation and Detection of Mycobacterium tuberculosis Complex and Nontuberculous Mycobacteria 5

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). The Taqman probes and the primers used in the detection of the target genes were designed using the Primer3 program.

(1) MTC

1) target gene: IS6110

2) primer

a. forward primer:
(SEQ ID NO: 58)
5′-cgaactcaaggagcacatcag-3′
b. reverse primer:
(SEQ ID NO: 59)
5′-gagtttggtcatcagccgttc-3′

3) Taqman probe

(SEQ ID NO: 21)
5′-HEX-cgccaactacggtgtttacggtg-BHQ1-3′
or
(SEQ ID NO: 60)
5′-VIC-agtgtggctaaccctgaac-MGB-3′

4) PCR product size: 136 bp

(2) NTM

1) target gene: 16S rRNA

2) primer

a. forward primer:
(SEQ ID NO: 61)
5′-tggcgaacgggtgagtaa-3′
b. reverse primer
(SEQ ID NO: 5)
5′-cccacaccgcaaaagctt-3′
(SEQ ID NO: 8)
5′-catcccacaccgctaccw-3′

3) Taqman probe

(SEQ ID NO: 62)
5′-FAM-cggtattagacccagtttcccagg-BHQ1-3′
or
(SEQ ID NO: 63)
5′-FAM-tgggaaactgggtctaatac-MGB-3′

4) PCR product size: 127 bp

### Example 5-1

### Duplex Real-Time PCR Using the Nucleotide Sequence of SEQ ID NO: 21 as a Taqman Probe for the Detection of MTC

(1) Isolation of DNA

DNA was isolated from 186 Mycobacterium species and 78 nontuberculous mycobacterium species, all recovered from clinical specimens, and from 68 standard mycobacteria species including M. abscessus ATCC 19977, M. acapulcensis KCTC 9501, M. africanum ATCC 25420, M. agri KCTC 9502, M. alvei KCTC 19709, M. asiaticum KCTC 9503, M. aurum KCTC 19457, M. austroafricanum KCTC 9504, M. avium ATCC 25291, M. bolletii KCTC 19281, M. botniense KCTC 19646, M. bovis ATCC 19210, M. brumae KCTC 19711, M. celatum ATCC 51131, M. chelonae subsp chelonae KCTC 9505, M. chlorophenolicum KCTC 19089, M. chubuense KCTC 19712, M. diernhoferi KCTC 9506, M. fallax KCTC 9508, M. flavescens ATCC 14474, M. fortuitum ATCC 6841, M. frederiksbergense KCTC 19100, M. gadium ATCC 27726, M. gastri ATCC 15754, M. gilvum KCTC 19423, M. goodii ATCC BAA-955, M. gordonae KCTC 9513, M. haemophilum ATCC 29548, M. hassiacum ATCC 700660, M. interjectum ATCC 51457, M. intermedium ATCC 51848, M. intracellulare ATCC 13950, M. intracellulare KCTC 9514, M. kansasii ATCC 12478, M. lentiflavum KMRC 70087, M. malmoense ATCC 29571, M. mantobense KCTC 9977, M. marinum ATCC 927, M. massiliense KCTC 19086, M. microti ATCC 19422, M. moriokaense KCTC 9516, M. mucogenicum KCTC 19088, M. neoaurum KCTC 19096, M. nonchromogenicum ATCC 19530, M. obuense KCTC 19097, M. parascrofulaceum KCTC 9979, M. peregrinum KCTC 9615, KMRC 75002, M. phlei KCTC 9689, M. porcinum KCTC 9517, M. pulveris KCTC 9518, M. scrofulaceum ATCC 19981, M. septicum ATCC 700731, M. simiae ATCC 25275, M. shimoidei ATCC 27962, M. smegmatis KCTC 9108, M. szulgai KCTC 9520, KMRC 31125, M. terrae KCTC 9614, M. triplex ATCC 700071, M. triviale KMRC 70093, M. tuberculosis ATCC 25177, ATCC 27294, M. ulcerans ATCC 19423, M. vaccae KCTC 19087, M. vanbaalenii KCTC 9966, M. wolinskyi ATCC 700010, and M. xenopi KMRC 42001.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and the KCTC species were cultured in broths while the KMRC species was grown on an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 15 sec, and annealing and elongation at 65° C. for 15 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 5, below. In the primer-probes Mix, the forward primer and the reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume as in MTC.

TABLE 5

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™, and Hex™ or VIC™ were monitored in a green channel (510±5 nm) and a yellow channel (555±5 nm), respectively.

### Example 5-2

### Duplex Real-Time PCR Using the Nucleotide Sequences of SEQ ID NOS: 21 and 63 as Taqman Probes for the Detection of MTC and NTM, Respectively

Duplex real-time PCR was carried out in the same manner as in Example 5-1, with the exception that the nucleotide sequences of SEQ ID NOS: 21 and 63 were used as Taqman probes for the detection of MTC and NTM, respectively.

### Example 5-3

### Duplex Real-Time PCR Using the Nucleotide Sequences of SEQ ID NOS: 60 and 62 as Taqman Probes for the Detection of MTC and NTM, Respectively

Duplex real-time PCR was carried out in the same manner as in Example 5-1, with the exception that the nucleotide sequences of SEQ ID NOS: 60 and 62 were used as Taqman probes for the detection of MTC and NTM, respectively.

### Example 5-4

### Duplex Real-Time PCR Using the Nucleotide Sequences of SEQ ID NOS: 60 and 63 as Taqman Probes for the Detection of MTC and NTM, Respectively

Duplex real-time PCR was carried out in the same manner as in Example 5-1, with the exception that the nucleotide sequences of SEQ ID NOS: 60 and 63 were used as Taqman probes for the detection of MTC and NTM, respectively.

2. Result of Duplex Real-Time PCR

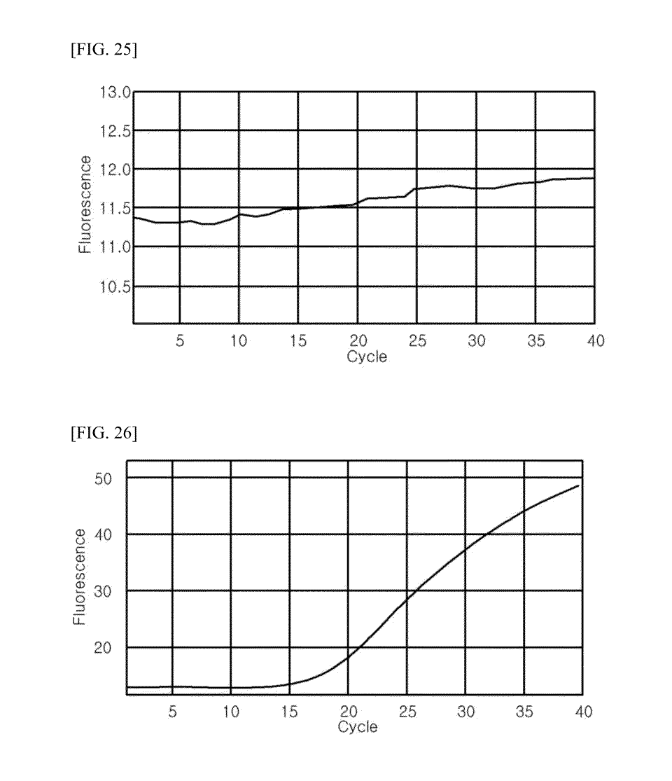

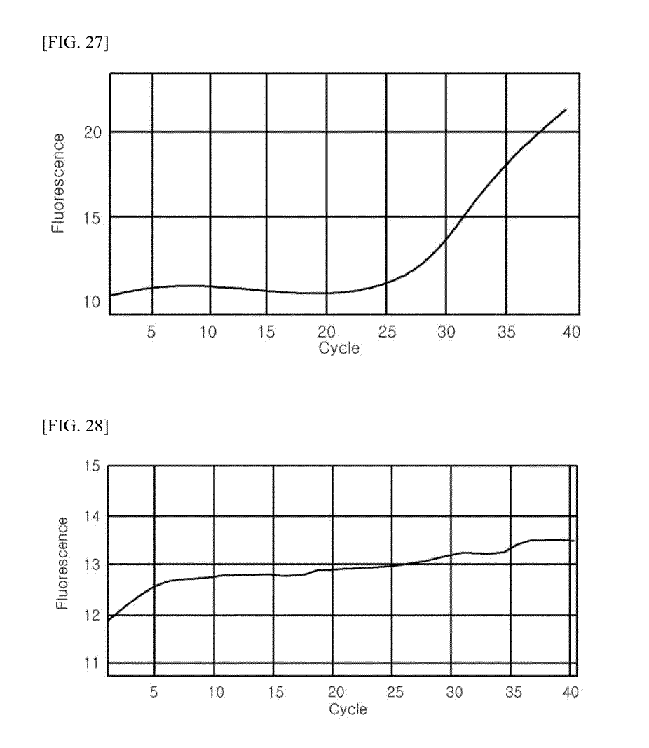

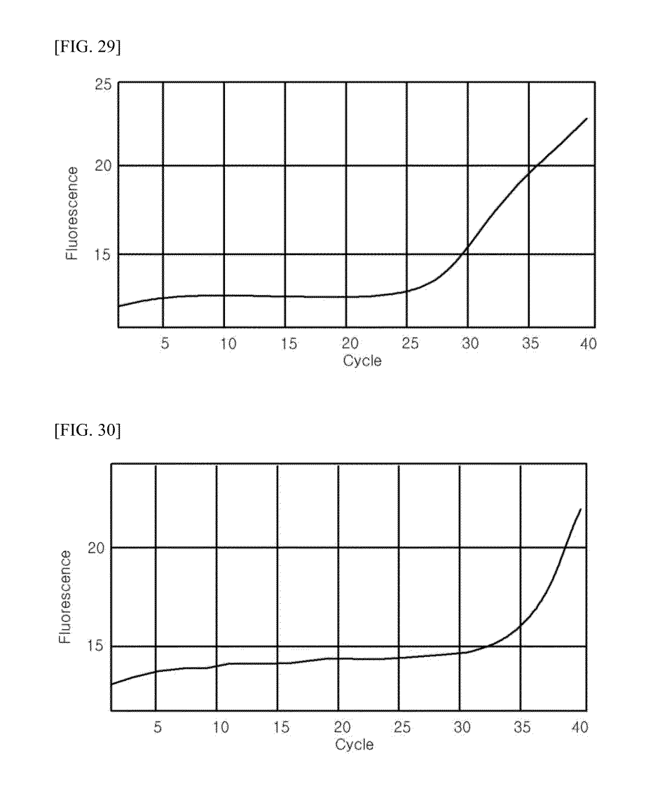

FIGS. 25 to 30 show results of the duplex real-time PCR performed with the mycobacteria. In the graphs, the number of cycles of PCR is set on the X-axis while fluorescence intensity (F) is on the Y-axis. FIGS. 25 and 26 are graphs in which fluorescence intensities in green and yellow channels are plotted, respectively, against the number of cycles of real-time PCR with MTC. FIGS. 27 and 28 are graphs in which fluorescence intensities in green and yellow channels are plotted, respectively, against the number of cycles of real-time PCR with NTM. FIGS. 29 and 30 are graphs in which fluorescence intensities in green and yellow channels are plotted, respectively, against the number of cycles of real-time PCR with MTC+NTM.

As can be seen in FIGS. 25 to 30, the IS6110 gene amplified from MTC was detected in the yellow channel and the 16S rRNA gene amplified from NTM in the green channel. Therefore, the duplex real-time PCR using the primers and the probes according to the present invention was found to guarantee the detection of MTC and NTM, simultaneously, with high reliability.

### Example 6

### Separation and Detection of Mycobacterium tuberculosis Complex and Nontuberculous Mycobacteria 6

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). The Taqman probes and the primers used in the detection of the target genes were designed using the Primer3 program.

(1) Mycobacterium tuberculosis complex (MTC)

1) target gene: IS6110

2) primer

a. forward primer:
(SEQ ID NO: 58)
5′-cgaactcaaggagcacatcag-3′
b. reverse primer:
(SEQ ID NO: 20)
5′-cagggttagccacactttgc-3′

3) Taqman probe

(SEQ ID NO: 21)
5′-Hex-cgccaactacggtgtttacggtg-BHQ1-3′
(SEQ ID NO: 64)
5′-VIC-ctacggtgtttacggtgc-MGB-3′

4) PCR product size: 79 bp

(2) Nontuberculous mycobacteria (NTM)

1) target gene: 16S rRNA

2) primer

a. forward primer
(SEQ ID NO: 65)
NTM-1: 5′-tktggtggaaagcttttgc-3′
(SEQ ID NO: 66)
NTM-2: 5′-ggtgwgtggtgcaaagctt-3′
(SEQ ID NO: 67)
NTM-3: 5′-tggtggaaagcgtttggt-3′
b. reverse primer:
(SEQ ID NO: 36)
5′-cgtaggagtctgggccgta-3′

3) Taqman probe

(SEQ ID NO: 39)
5′-FAM-cgggtagccggcctgagag-BHQ1-3′
or
(SEQ ID NO: 68)
5′-FAM-cctgagagggtgwccg-MGB-3′

4) PCR product size: 146 bp˜148 bp

### Example 6-1

### Duplex Real-Time PCR Using the Nucleotide Sequences of SEQ ID NOS: 21 and 39 as Taqman Probes for the Detection of MTC and NTM, Respectively

(1) Isolation of DNA

DNA was isolated from 186 Mycobacterium species and 78 nontuberculous mycobacterium species, all recovered from clinical specimens, and from 68 standard mycobacteria species. These standard species were the same as in Example 5-1.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and the KCTC species were cultured in broths while the KMRC species was grown in an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 15 sec, and annealing and elongation at 64° C. for 15 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 6, below. In the primer-probes Mix, the forward primer and the reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume as in MTC.

TABLE 6

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| Primers-Probes Mix | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™, and Hex™ or VIC™ were monitored in a green channel (510±5 nm) and a yellow channel (555±5 nm), respectively.

### Example 6-2

### Duplex Real-Time PCR Using the Nucleotide Sequences of SEQ ID NOS: 21 and 68 as Taqman Probes for the Detection of MTC and NTM, Respectively

Duplex real-time PCR was carried out in the same manner as in Example 6-1, with the exception that the nucleotide sequences of SEQ ID NOS: 21 and 68 were used as Taqman probes for the detection of MTC and NTM, respectively.

### Example 6-3

### Duplex Real-Time PCR Using the Nucleotide Sequences of SEQ ID NOS: 64 and 39 as Taqman Probes for the Detection of MTC and NTM, Respectively

Duplex real-time PCR was carried out in the same manner as in Example 6-1, with the exception that the nucleotide sequences of SEQ ID NOS: 64 and 39 were used as Taqman probes for the detection of MTC and NTM, respectively.

### Example 6-4

### Duplex Real-Time PCR Using the Nucleotide Sequences of SEQ ID NOS: 64 and 68 as Taqman Probes for the Detection of MTC and NTM, Respectively

Duplex real-time PCR was carried out in the same manner as in Example 6-1, with the exception that the nucleotide sequences of SEQ ID NOS: 64 and 68 were used as Taqman probes for the detection of MTC and NTM, respectively.

2. Result of Duplex Real-Time PCR

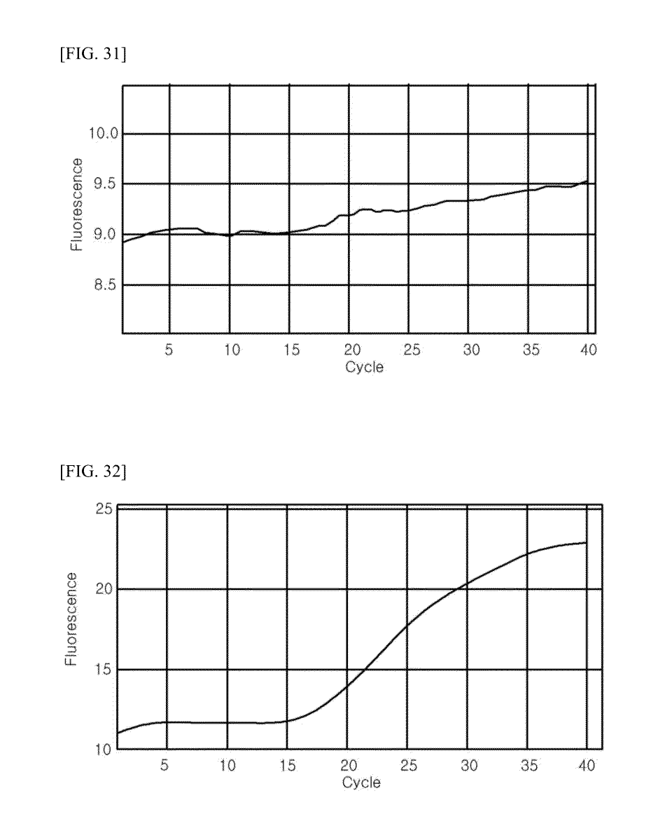

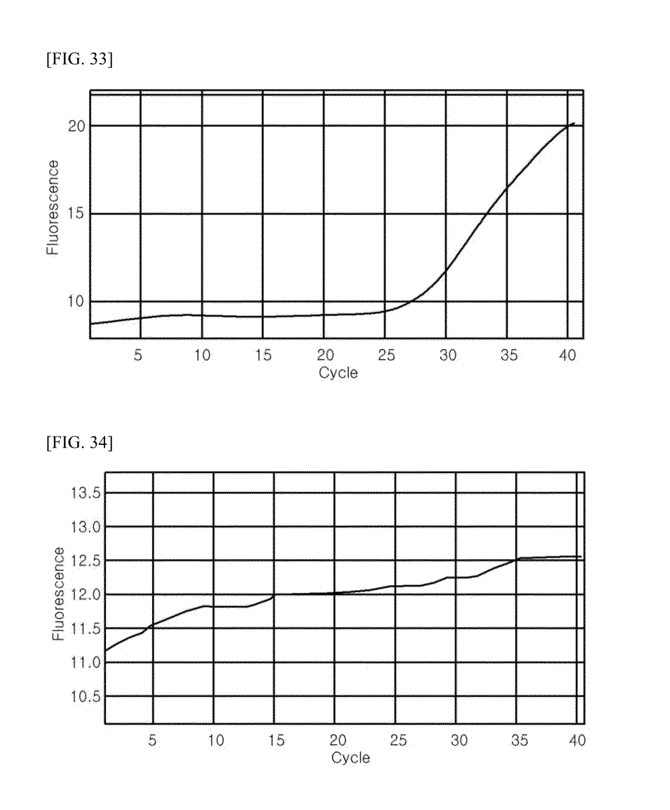

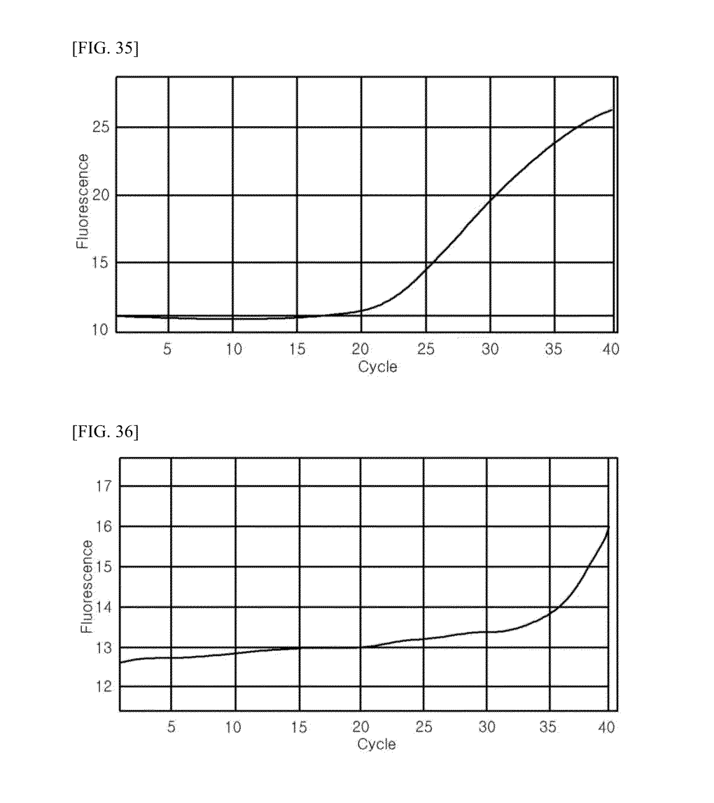

FIGS. 31 to 36 show results of the duplex real-time PCR performed with the mycobacteria. In the graphs, the number of cycles of PCR is set on the X-axis while fluorescence intensity (F) is on the Y-axis. FIGS. 31 and 32 are graphs in which fluorescence intensities in green and yellow channels are plotted, respectively, against the number of cycles of real-time PCR with MTC. FIGS. 33 and 34 are graphs in which fluorescence intensities in green and yellow channels are plotted, respectively, against the number of cycles of real-time PCR with NTM. FIGS. 35 and 36 are graphs in which fluorescence intensities in green and yellow channels are plotted, respectively, against the number of cycles of real-time PCR with MTC+NTM.

As can be seen in FIGS. 31 to 36, the IS6110 gene amplified from MTC was detected in the yellow channel and the 16S rRNA gene amplified from NTM in the green channel. Therefore, the duplex real-time PCR using the primers and the probes according to the present invention was found to guarantee the detection of MTC and NTM, simultaneously, with high reliability.

### Example 7

### Separation and Detection of Mycobacterium tuberculosis Complex and Nontuberculous Mycobacteria 7

1. Detection Target and Primer Design

Target genes to be detected were the IS6110 gene for Mycobacterium tuberculosis complex (MTC: M. tuberculosis, M. bovis, M. africanum, M. microti), and the 16S rRNA gene for nontuberculous mycobacteria (NTM). Nucleotide sequences of the 16S rRNA gene of NTM were used as Taqman probes. Universal primers were used to amplify the 16S rRNA gene of mycobacteria. The primers useful for the amplification of target genes were designed using the Primer3 program.

(1) Mycobacterium tuberculosis complex (MTC)

1) target gene: IS6110

2) primer

a. forward primer:
(SEQ ID NO: 58)
5′-cgaactcaaggagcacatcag-3′
b. reverse primer:
(SEQ ID NO: 59)
5′-gagtttggtcatcagccgttc-3′

3) Taqman probe

(SEQ ID NO: 60)
5′-VIC-agtgtggctaaccctgaac-MGB-3′

4) PCR product size: 136 bp

(2) Nontuberculous mycobacteria (NTM)

1) target gene: 16S rRNA

2) primer

a. forward primer:
(SEQ ID NO: 24)
5′-ggataagcytgggaaactgg-3′
b. reverse primer:
(SEQ ID NO: 75)
5′-cgtaggagtctgggccgta-3′

3) Taqman probe

NTM-1:
(SEQ ID NO: 27)
5′-FAM-tggtggaaagcttttgc-MGB-3′
NTM-2:
(SEQ ID NO: 76)
5′-FAM-ccacaccgctaccaaac-MGB-3′

4) PCR product size: 205 bp

2. Duplex real-time PCR

(1) Isolation of DNA

DNA was isolated from 186 Mycobacterium species and 78 nontuberculous mycobacterium species, all recovered from clinical specimens, and from 68 standard mycobacteria species. These standard species were the same as in Example 5-1.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and the KCTC species were cultured in broths while the KMRC species was grown in an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for 10 min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex Real-Time PCR

Duplex real-time PCR started with denaturation at 95° C. for 5 min and was run with 40 cycles of denaturation at 95° C. for 15 sec, and annealing and elongation at 65° C. for 15 sec, using a Rotor-Gene multiplex PCR Kit (QIAGEN Inc., Germantown, Md., USA) on Rotor-Gene Q (QIAGEN Inc., Germantown, Md., USA). The composition of the duplex real-time PCR reagent is summarized in Table 7, below. In the primer-probes Mix, the forward primer and the reverse primer were contained in the same amounts (10 pmoles/μL), with a probe present in an amount of 4 pmoles/μL. Accordingly, 1.25 μL of the primer-probes mix for MTC contained the forward primer and the reverse primer in an amount of 12.5 pmoles, each and the probe in an amount of 5 pmoles. Since a total volume of PCR mixture was 25 μL, it contained the primers at a concentration of 0.5 μM (12.5 pmoles/25 μL), and the probe at a concentration of 0.2 μM (5 pmoles/25 μL). For NTM, the forward primer, the reverse primer, and the probe were used in the same concentration and volume as in MTC.

TABLE 7

| Ingredient | | Vol. (μL) | Conc. |
|---|---|---|---|
| 2X Rotor-Gene Multiplex PCR Master Mix | | 12.5 | 1X |
| Primers-Probes Mix | Primer (10 pmoles/μL) | 1.25 | 0.5 μM |
| | Probe (4 pmoles/μL) | 1.25 | 0.2 μM |

| | | |
|---|---|---|
| Nuclease free water | 6.25 | − |
| Sample DNA template | 5 | − |
| Total | 25 | − |

The duplex real-time PCR relied upon the detection and quantitation of a fluorescent reporter, the signal of which was formed in the annealing and the elongation step on Rotor-Gene Q (QIAGEN Inc., Germantown, Md., USA). In the duplex real-time PCR, the fluorescent signal was detected and quantitated in real-time in each cycle of PCR on the basis of fluorescence resonance energy transfer (FRET). Fluorescent signals of FAM™, and VIC™ were monitored in a green channel (510±5 nm) and a yellow channel (555±5 nm), respectively.

3. Result of Duplex Real-Time PCR

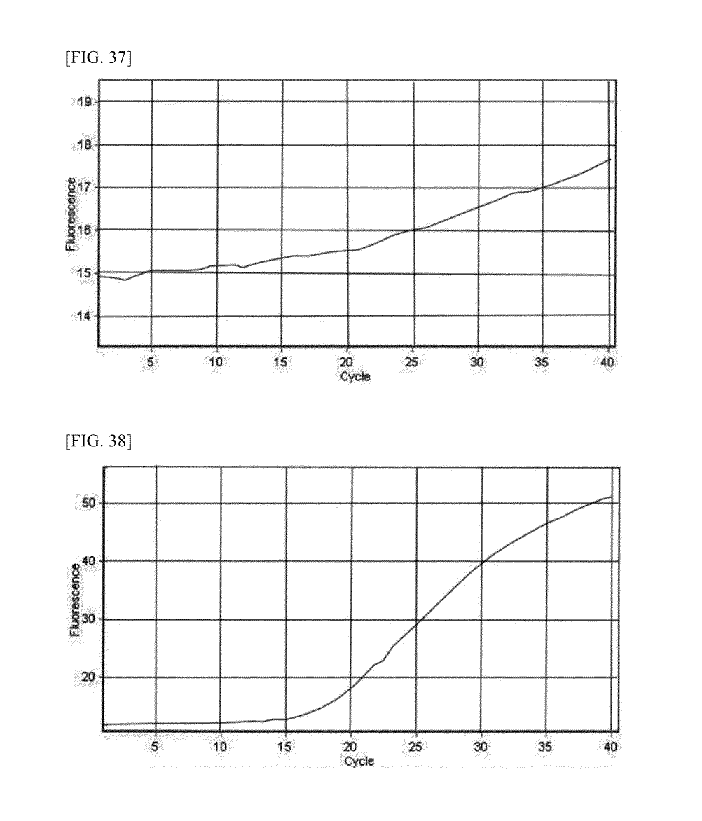

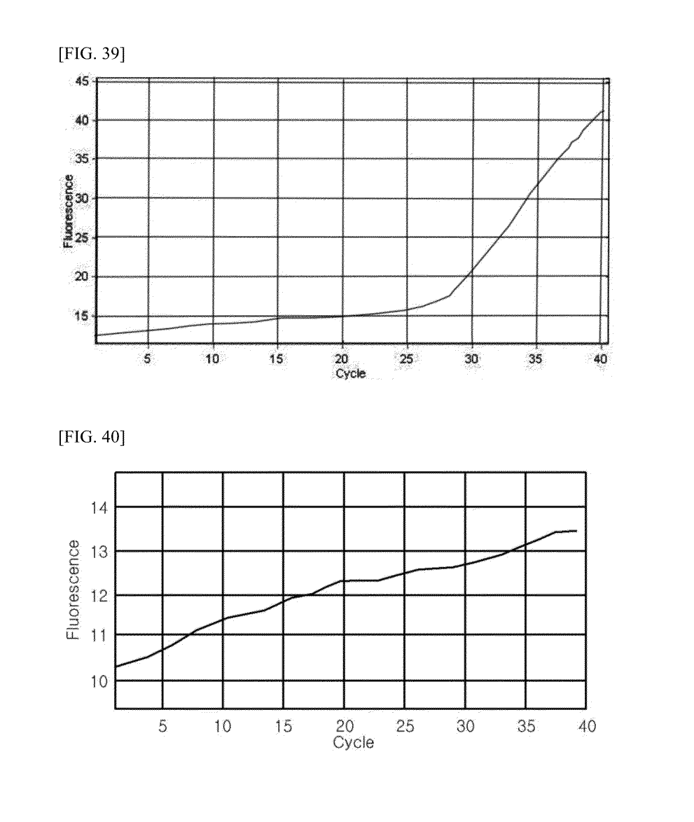

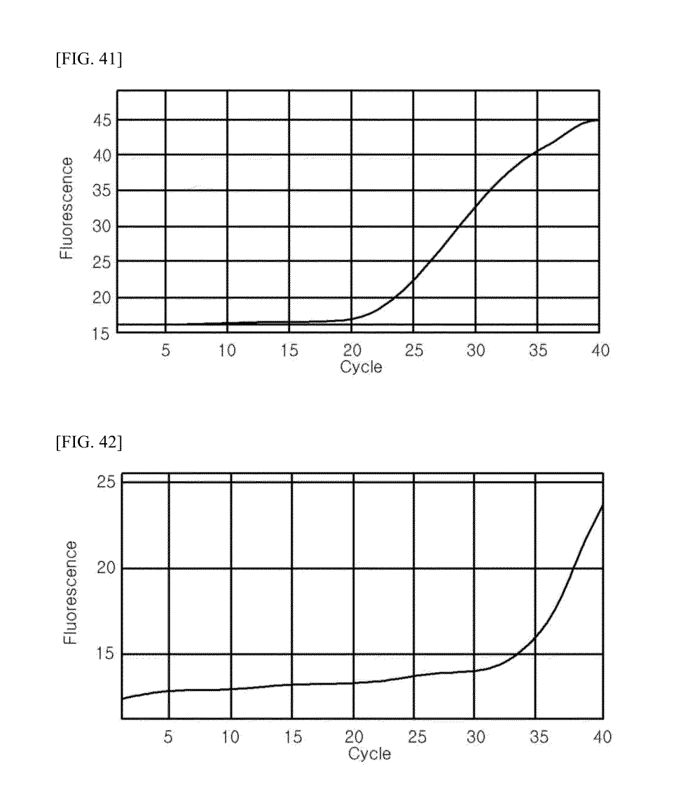

FIGS. 37 to 42 show results of the duplex real-time PCR performed with the mycobacteria. In the graphs, the number of cycles of PCR is set on the X-axis while fluorescence intensity (F) is on the Y-axis. FIGS. 37 and 38 are graphs in which fluorescence intensities in green and yellow channels are plotted, respectively, against the number of cycles of real-time PCR with MTC. FIGS. 39 and 40 are graphs in which fluorescence intensities in green and yellow channels are plotted, respectively, against the number of cycles of real-time PCR with NTM. FIGS. 41 and 42 are graphs in which fluorescence intensities in green and yellow channels are plotted, respectively, against the number of cycles of real-time PCR with MTC+NTM.

As can be seen in FIGS. 37 to 42, the IS6110 gene amplified from MTC was detected in the yellow channel and the 16S rRNA gene amplified from NTM in the green channel. Therefore, the duplex real-time PCR using the primers and the probes according to the present invention was found to guarantee the detection of MTC and NTM, simultaneously, with high reliability.

In combination with detection kits comprising forward and reverse primers and/or probes designed on the basis of nucleotide sequences which are characteristic of MTC, or which are absent from MTC but intrinsic to NTM, as demonstrated in the Examples, duplex real-time PCR can be used to detect MTC and NTM with high reliability. Therefore, the present invention provides a means for detecting MTC and NTM effectively.

Being capable of detecting nucleotide sequences characteristic of MTC and NTM, the primers and/or probes of the present invention are highly sensitive to and selective for MTC and NTM when applied to detection, as described above. In addition, the duplex real-time PCR using the primers and/or probes according to the present invention is a clinical diagnostic means that promises the very effective, simultaneous detection of both MTC and NTM in a target subject.

### INDUSTRIAL APPLICABILITY

Useful in detecting genes characteristic of MTC and NTM, as described hitherto, the primer sets and/or probes, detection kits, and detection methods according to the present invention can be applied to the clinical diagnosis of diseases caused by MTC and NTM, and therefore find applications in the medical fields including hospitals, research institutes, etc.

### SEQUENCE LIST TEXT

The nucleotide sequence of SEQ ID NO: 1 is a forward primer specific for the IS6110 gene of Mycobacterium tuberculosis complex.

The nucleotide sequence of SEQ ID NO: 2 is a reverse primer specific for the IS6100 gene of Mycobacterium tuberculosis complex.

The nucleotide sequence of SEQ ID NO: 3 is a probe specific for the IS6110 gene of Mycobacterium tuberculosis complex.

The nucleotide sequence of SEQ ID NO: 4 is a forward primer specific for the 16S rRNA gene of nontuberculous mycobacteria.

The nucleotide sequence of SEQ ID NO: 5 is a reverse primer (NTM-1) specific for the 16S rRNA gene of nontuberculous mycobacteria.

The nucleotide sequence of SEQ ID NO: 6 is a reverse primer (NTM-1) specific for the 16S rRNA gene of nontuberculous mycobacteria.

The nucleotide sequence of SEQ ID NO: 7 is a reverse primer (NTM-1) specific for the 16S rRNA gene of nontuberculous mycobacteria.

The nucleotide sequence of SEQ ID NO: 8 is a reverse primer (NTM-2) specific for the 16S rRNA gene of nontuberculous mycobacteria.

The nucleotide sequence of SEQ ID NO: 9 is a probe specific for the 16S rRNA gene of nontuberculous mycobacteria.

The nucleotide sequence of SEQ ID NO: 10 is a reverse primer (NTM-2) specific for the 16S rRNA gene of nontuberculous mycobacteria.

The nucleotide sequence of SEQ ID NO: 11 is a reverse primer (NTM-2) specific for the 16S rRNA gene of nontuberculous mycobacteria.

The nucleotide sequence of SEQ ID NO: 12 is a forward primer specific for the 16S rRNA gene of nontuberculous mycobacteria.

The nucleotide sequence of SEQ ID NO: 13 is a forward primer specific for the 16S rRNA gene of nontuberculous mycobacteria.

The nucleotide sequence of SEQ ID NO: 14 is a forward primer specific for the 16S rRNA gene of nontuberculous mycobacteria.

The nucleotide sequence of SEQ ID NO: 15 is a forward primer specific for the 16S rRNA gene of nontuberculous mycobacteria.

The nucleotide sequence of SEQ ID NO: 16 is a forward primer specific for the 16S rRNA gene of nontuberculous mycobacteria.

The nucleotide sequence of SEQ ID NO: 17 is a forward primer specific for the 16S rRNA gene of nontuberculous mycobacteria.

The nucleotide sequence of SEQ ID NO: 18 is a forward primer specific for the 16S rRNA gene of nontuberculous mycobacteria.

The nucleotide sequence of SEQ ID NO: 19 is a forward primer specific for the 16S rRNA gene of nontuberculous mycobacteria.

The nucleotide sequence of SEQ ID NO: 20 is a reverse primer specific for the IS6110 gene of Mycobacterium tuberculosis complex.

The nucleotide sequence of SEQ ID NO: 21 is a probe specific for the IS6110 gene of Mycobacterium tuberculosis complex.

The nucleotide sequence of SEQ ID NO: 22 is a forward primer specific for the 16S rRNA gene of nontuberculous mycobacteria.

The nucleotide sequence of SEQ ID NO: 23 is a probe specific for the 16S rRNA gene of nontuberculous mycobacteria.

The nucleotide sequence of SEQ ID NO: 24 is a forward universal primer specific for the 16S rRNA gene of Mycobacterium.

The nucleotide sequence of SEQ ID NO: 25 is a reverse universal primer specific for the 16S rRNA gene of Mycobacterium.

The nucleotide sequence of SEQ ID NO: 26 is a probe specific for the 16S rRNA specific for the 16S rRNA of Mycobacterium tuberculosis complex.

The nucleotide sequence of SEQ ID NO: 27 is a probe (NTM-1) specific for the 16S rRNA gene of nontuberculous mycobacteria.

The nucleotide sequence of SEQ ID NO: 28 is a probe (NTM-2) specific for the 16S rRNA gene of nontuberculous mycobacteria.

The nucleotide sequence of SEQ ID NO: 29 is a forward universal primer specific for the 16S rRNA gene of Mycobacterium.

The nucleotide sequence of SEQ ID NO: 30 is a forward universal primer specific for the 16S rRNA gene of Mycobacterium.

The nucleotide sequence of SEQ ID NO: 31 is a reverse universal primer specific for the 16S rRNA gene of Mycobacterium.

The nucleotide sequence of SEQ ID NO: 32 is a reverse universal primer specific for the 16S rRNA gene of Mycobacterium.

The nucleotide sequence of SEQ ID NO: 33 is probe (NTM-2) specific for the 16S rRNA gene of nontuberculous mycobacteria.

The nucleotide sequence of SEQ ID NO: 34 is a probe (NTM-2) specific for the 16S rRNA gene of nontuberculous mycobacteria.

The nucleotide sequence of SEQ ID NO: 35 is a forward primer specific for the 16S rRNA gene of nontuberculous mycobacteria.

The nucleotide sequence of SEQ ID NO: 36 is a reverse primer specific for the 16S rRNA gene of nontuberculous mycobacteria.

The nucleotide sequence of SEQ ID NO: 37 is a probe specific for the 16S rRNA gene of nontuberculous mycobacteria.

The nucleotide sequence of SEQ ID NO: 38 is a probe specific for the 16S rRNA gene of nontuberculous mycobacteria.

The nucleotide sequence of SEQ ID NO: 39 is a probe specific for the 16S rRNA gene of nontuberculous mycobacteria.

The nucleotide sequence of SEQ ID NO: 40 is a forward primer specific for the 16S rRNA gene of nontuberculous mycobacteria.

The nucleotide sequence of SEQ ID NO: 41 is a forward primer specific for the 16S rRNA gene of nontuberculous mycobacteria.

The nucleotide sequence of SEQ ID NO: 42 is a forward primer specific for the 16S rRNA gene of nontuberculous mycobacteria.

The nucleotide sequence of SEQ ID NO: 43 is a forward primer specific for the 16S rRNA gene of nontuberculous mycobacteria.

The nucleotide sequence of SEQ ID NO: 44 is a forward primer specific for the 16S rRNA gene of nontuberculous mycobacteria.

The nucleotide sequence of SEQ ID NO: 45 is a forward primer specific for the 16S rRNA gene of nontuberculous mycobacteria.

The nucleotide sequence of SEQ ID NO: 46 is a forward primer specific for the 16S rRNA gene of nontuberculous mycobacteria.

The nucleotide sequence of SEQ ID NO: 47 is a forward primer specific for the 16S rRNA gene of nontuberculous mycobacteria.

The nucleotide sequence of SEQ ID NO: 48 is a forward primer specific for the 16S rRNA gene of nontuberculous mycobacteria.

The nucleotide sequence of SEQ ID NO: 49 is a forward primer specific for the 16S rRNA gene of nontuberculous mycobacteria.

The nucleotide sequence of SEQ ID NO: 50 is a forward primer specific for the 16S rRNA gene of nontuberculous mycobacteria.

The nucleotide sequence of SEQ ID NO: 51 is a forward primer specific for the 16S rRNA gene of nontuberculous mycobacteria.

The nucleotide sequence of SEQ ID NO: 52 is a forward primer specific for the 16S rRNA gene of nontuberculous mycobacteria.

The nucleotide sequence of SEQ ID NO: 53 is a forward primer specific for the 16S rRNA gene of nontuberculous mycobacteria.

The nucleotide sequence of SEQ ID NO: 54 is a forward primer specific for the 16S rRNA gene of nontuberculous mycobacteria.

The nucleotide sequence of SEQ ID NO: 55 is a forward primer specific for the 16S rRNA gene of nontuberculous mycobacteria.

The nucleotide sequence of SEQ ID NO: 56 is a probe specific for the 16S rRNA gene of nontuberculous mycobacteria.

The nucleotide sequence of SEQ ID NO: 57 is a probe specific for the 16S rRNA gene of nontuberculous mycobacteria.

The nucleotide sequence of SEQ ID NO: 58 is a forward primer specific for the IS6110 gene of Mycobacterium tuberculosis complex.

The nucleotide sequence of SEQ ID NO: 59 is a reverse primer specific for the IS6110 gene of Mycobacterium tuberculosis complex.

The nucleotide sequence of SEQ ID NO: 60 is a probe specific for the IS6110 gene of Mycobacterium tuberculosis complex.

The nucleotide sequence of SEQ ID NO: 61 is a forward primer specific for the 16S rRNA gene of nontuberculous mycobacteria.

The nucleotide sequence of SEQ ID NO: 62 is a probe specific for the 16S rRNA gene of nontuberculous mycobacteria.

The nucleotide sequence of SEQ ID NO: 63 is a probe specific for the 16S rRNA gene of nontuberculous mycobacteria.

The nucleotide sequence of SEQ ID NO: 64 is a probe specific for the IS6110 gene of Mycobacterium tuberculosis complex.

The nucleotide sequence of SEQ ID NO: 65 is a forward primer (NTM-1) specific for the 16S rRNA gene of nontuberculous mycobacteria.

The nucleotide sequence of SEQ ID NO: 66 is a forward primer (NTM-2) specific for the 16S rRNA gene of nontuberculous mycobacteria.

The nucleotide sequence of SEQ ID NO: 67 is a forward primer (NTM-3) specific for the 16S rRNA gene of nontuberculous mycobacteria.

The nucleotide sequence of SEQ ID NO: 68 is a probe specific for the 16S rRNA gene of nontuberculous mycobacteria.

The nucleotide sequence of SEQ ID NO: 69 is a forward primer (NTM-1) specific for the 16S rRNA gene of nontuberculous mycobacteria.

The nucleotide sequence of SEQ ID NO: 70 is a forward primer (NTM-1) specific for the 16S rRNA gene of nontuberculous mycobacteria.

The nucleotide sequence of SEQ ID NO: 71 is a forward primer (NTM-2) specific for the 16S rRNA gene of nontuberculous mycobacteria.

The nucleotide sequence of SEQ ID NO: 72 is a forward primer (NTM-2) specific for the 16S rRNA gene of nontuberculous mycobacteria.

The nucleotide sequence of SEQ ID NO: 73 is a probe specific for the 16S rRNA gene of nontuberculous mycobacteria.

The nucleotide sequence of SEQ ID NO: 74 is a probe specific for the 16S rRNA gene of nontuberculous mycobacteria.

The nucleotide sequence of SEQ ID NO: 75 is a reverse universal primer specific for the 16S rRNA gene of Mycobacterium.

The nucleotide sequence of SEQ ID NO: 76 is a probe (NTM-2) specific for the 16S rRNA gene of nontuberculous mycobacteria.

1. A probe for detecting a 16S rRNA gene of nontuberculous mycobacteria, having a nucleotide sequence of SEQ ID NO: 9.

2. The probe of claim 1, wherein the probe is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ (molecular grove binding non-fluorescence quencher).

3. A kit for detection of Mycobacterium tuberculosis and nontuberculous mycobacteria, comprising:
a primer set specific for an IS6110 gene of Mycobacterium tuberculosis, composed of a forward primer having a nucleotide sequence of SEQ ID NO: 1 and a reverse primer having a nucleotide sequence of SEQ ID NO: 2;
a probe, having a nucleotide sequence of SEQ ID NO: 3, for detecting the IS6110 gene of Mycobacterium tuberculosis;
a primer set specific for a 16S rRNA gene of nontuberculous mycobacteria, composed of a forward primer having a nucleotide sequence of SEQ ID NO: 4, at least one reverse primer selected from the group consisting of nucleotide sequences of SEQ ID NOS: 5 to 7, and a reverse primer having a nucleotide sequence of SEQ ID NO: 8; and
a probe, having a nucleotide sequence of SEQ ID NO: 9, for detecting the 16S rRNA gene of nontuberculous mycobacteria.

4. The kit of claim 3, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

5. A method for detecting Mycobacterium tuberculosis and nontuberculous mycobacteria, comprising:
isolating DNA from a test subject;
amplifying the DNA by duplex real-time PCR using a primer set specific for a IS6110 gene of Mycobacterium tuberculosis, consisting of a forward primer having a nucleotide sequence of SEQ ID NO: 1 and a reverse primer having a nucleotide sequence of SEQ ID NO: 2, a probe, having a nucleotide sequence of SEQ ID NO: 3, for detecting the IS6110 gene of Mycobacterium tuberculosis, a primer set specific for a 16S rRNA gene of nontuberculous mycobacteria, composed of a forward primer having a nucleotide sequence of SEQ ID NO: 4, at least one reverse primer selected from the group consisting of nucleotide sequences of SEQ ID NOS: 5 to 7 and a reverse primer having a nucleotide sequence of SEQ ID NO: 8, and a probe, having a nucleotide sequence of SEQ ID NO: 9, for detecting the 16S rRNA gene of nontuberculous mycobacteria; and
analyzing products of the duplex real-time PCR.

6. A primer set specific for a 16S rRNA gene of nontuberculous mycobacteria, comprising:
a forward primer having a nucleotide sequence of SEQ ID NO: 22,
at least one reverse primer selected from the group consisting of nucleotide sequences of SEQ ID NOS: 5 to 7, and
a reverse primer having a nucleotide sequence of SEQ ID NO: 8.

7. A probe for detecting a 16S rRNA gene of nontuberculous mycobacteria, having a nucleotide sequence of SEQ ID NO: 23.

8. The probe of claim 7, wherein the probe is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, and BHQ-1,2,3.

9. A kit for detection of Mycobacterium tuberculosis and nontuberculous mycobacteria, comprising:
a primer set specific for an IS6110 gene of Mycobacterium tuberculosis, composed of a forward primer having a nucleotide sequence of SEQ ID NO: 1 and a reverse primer having a nucleotide sequence of SEQ ID NO: 20;
a probe, having a nucleotide sequence of SEQ ID NO: 21, for detecting the IS6110 gene of Mycobacterium tuberculosis;
a primer set specific for a 16S rRNA gene of nontuberculous mycobacteria, composed of a forward primer having a nucleotide sequence of SEQ ID NO: 22, at least one reverse primer selected from the group consisting of nucleotide sequences of SEQ ID NOS: 5 to 7, and a reverse primer having a nucleotide sequence of SEQ ID NO: 8; and
a probe, having a nucleotide sequence of SEQ ID NO: 23, for detecting the 16S rRNA gene of nontuberculous mycobacteria.

10. The kit of claim 9, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

11. A method for detecting Mycobacterium tuberculosis and nontuberculous mycobacteria, comprising:
isolating DNA from a test subject;
amplifying the DNA by duplex real-time PCR using a primer set specific for an IS6110 gene of Mycobacterium tuberculosis, consisting of a forward primer having a nucleotide sequence of SEQ ID NO: 1 and a reverse primer having a nucleotide sequence of SEQ ID NO: 20, a probe, having a nucleotide sequence of SEQ ID NO: 21, for detecting the IS6110 gene of Mycobacterium tuberculosis, a primer set specific for a 16S rRNA gene of nontuberculous mycobacteria, composed of a forward primer having a nucleotide sequence of SEQ ID NO: 22, at least one reverse primer selected from the group consisting of nucleotide sequences of SEQ ID NOS: 5 to 7, and a reverse primer having a nucleotide sequence of SEQ ID NO: 8, and a probe, having a nucleotide sequence of SEQ ID NO: 23, for detecting the 16S rRNA gene of nontuberculous mycobacteria; and
analyzing products of the duplex real-time PCR.

12. A probe for detecting a 16S rRNA gene of Mycobacterium tuberculosis, having a nucleotide sequence of SEQ ID NO: 26.

13. The probe of claim 12, wherein the probe is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ.

14. A probe for detecting a 16S rRNA of nontuberculous mycobacterium, comprising a probe having a nucleotide sequence of SEQ ID NO: 27 and a probe having a nucleotide sequence of SEQ ID NO: 28.

15. The probe of claim 14, wherein the probe is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ.

16. A kit for detection of Mycobacterium tuberculosis and nontuberculous mycobacteria, comprising:
a universal primer set for amplifying 16S rRNA genes of Mycobacterium tuberculosis and nontuberculous mycobacterium, composed of a forward primer having a nucleotide sequence of SEQ ID NO: 24 and a reverse primer having a nucleotide sequence of SEQ ID NO: 25;
a probe for detecting the 16S rRNA gene of Mycobacterium tuberculosis, having a nucleotide sequence of SEQ ID NO: 26; and
a probe for detecting the 16S rRNA gene of nontuberculous mycobacteria, composed of a probe having a nucleotide sequence of SEQ ID NO: 27 and a probe having a nucleotide sequence of SEQ ID NO: 28.

17. The kit of claim 16, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

18. A method for detecting Mycobacterium tuberculosis and nontuberculous mycobacteria, comprising:
isolating DNA from a test subject;
amplifying the DNA by duplex real-time PCR using a universal primer set for amplifying 16S rRNA genes of Mycobacterium tuberculosis and nontuberculous mycobacterium, composed of a forward primer having a nucleotide sequence of SEQ ID NO: 24 and a reverse primer having a nucleotide sequence of SEQ ID NO: 25, a probe for detecting a 16S rRNA gene of Mycobacterium tuberculosis, having the nucleotide sequence of SEQ ID NO: 26; and a probe for detecting the 16S rRNA gene of nontuberculous mycobacteria, composed of a probe having a nucleotide sequence of SEQ ID NO: 27 and a probe having a nucleotide sequence of SEQ ID NO: 28; and
analyzing products of the duplex real-time PCR.

19. A probe for detecting a 16S rRNA gene of nontuberculous mycobacteria, having one selected from the group consisting of nucleotide sequences of SEQ ID NOS: 37 to 39.

20. The probe of claim 19, wherein the probe is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ.

21. A kit for detection of Mycobacterium tuberculosis and nontuberculous mycobacteria, comprising:
a primer set specific for an IS6110 gene of Mycobacterium tuberculosis, consisting of a forward primer having a nucleotide sequence of SEQ ID NO: 1 and a reverse primer having a nucleotide sequence of SEQ ID NO: 20;
a probe, having a nucleotide sequence of SEQ ID NO: 21, for detecting the IS6110 gene of Mycobacterium tuberculosis;
a primer set specific for a 16S rRNA gene of nontuberculous mycobacteria, composed of a forward primer having a nucleotide sequence of SEQ ID NO: 35, and a reverse primer having a nucleotide sequence of SEQ ID NO: 36; and
a probe for detecting the 16S rRNA gene of nontuberculous mycobacteria, having one selected from the group consisting of nucleotides sequences of SEQ ID NOS: 37 to 39.

22. The kit of claim 21, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

23. A method for detecting Mycobacterium tuberculosis and nontuberculous mycobacteria, comprising:
isolating DNA from a test subject;
amplifying the DNA by duplex real-time PCR using a primer set specific for an IS6110 gene of Mycobacterium tuberculosis, consisting of a forward primer having a nucleotide sequence of SEQ ID NO: 1 and a reverse primer having a nucleotide sequence of SEQ ID NO: 20, a probe, having a nucleotide sequence of SEQ ID NO: 21, for detecting the IS6110 gene of Mycobacterium tuberculosis, a primer set specific for a 16S rRNA gene of nontuberculous mycobacteria, composed of a forward primer having a nucleotide sequence of SEQ ID NO: 35, and a reverse primer having a nucleotide sequence of SEQ ID NO: 36, and a probe for detecting the 16S rRNA gene of nontuberculous mycobacteria, having one selected from the group consisting of nucleotides sequences of SEQ ID NOS: 37 to 39; and
analyzing products of the duplex real-time PCR.

24. A primer set specific for a 16S rRNA gene of nontuberculous mycobacteria, comprising:
a forward primer having a nucleotide sequence of SEQ ID NO: 61; and
a reverse primer composed of a primer having a nucleotide sequence of SEQ ID NO: 5 and a primer having a nucleotide sequence of SEQ ID NO: 8.

25. A kit for detection of Mycobacterium tuberculosis and nontuberculous mycobacteria, comprising:
a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 59;
a probe for detecting the IS6110 gene of Mycobacterium tuberculosis, having a nucleotide sequence of SEQ ID NO: 21 or SEQ ID NO: 60;
a primer set specific for a 16S rRNA gene of nontuberculous mycobacteria, comprising a forward primer having a nucleotide sequence of SEQ ID NO: 61, and a reverse primer composed of a primer having a nucleotide sequence of SEQ ID NO: 5 and a primer having a nucleotide sequence of SEQ ID NO: 8; and
a probe for detecting the 16S rRNA gene of nontuberculous mycobacteria, having a nucleotide sequence of SEQ ID NO: 62 or 63.

26. The kit of claim 25, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

27. A method for detecting Mycobacterium tuberculosis and nontuberculous mycobacteria, comprising:
isolating DNA from a test subject;
amplifying the DNA by duplex real-time PCR using a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 59, a probe for detecting the IS6110 gene of Mycobacterium tuberculosis, having a nucleotide sequence of SEQ ID NO: 21 or SEQ ID NO: 60, a primer set specific for a 16S rRNA gene of nontuberculous mycobacteria, comprising a forward primer having a nucleotide sequence of SEQ ID NO: 61, and a reverse primer composed of a primer having a nucleotide sequence of SEQ ID NO: 5 and a primer having a nucleotide sequence of SEQ ID NO: 8; and a probe for detecting the 16S rRNA gene of nontuberculous mycobacteria, having a nucleotide sequence of SEQ ID NO: 62 or 63; and
analyzing products of the duplex real-time PCR.

28. A primer set specific for a 16S rRNA gene of nontuberculous mycobacteria, comprising:
a forward primer comprising a primer having a nucleic acid sequence of SEQ ID NO: 65, a primer having a nucleotide sequence of SEQ ID NO: 66, and a primer having a nucleotide sequence of SEQ ID NO: 67; and
a reverse primer comprising a nucleotide sequence of SEQ ID NO: 36.

29. A kit for detection of Mycobacterium tuberculosis and nontuberculous mycobacteria, comprising:
a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 20;
a probe for detecting the IS6110 gene of Mycobacterium tuberculosis, having a nucleotide sequence of SEQ ID NO: 21 or SEQ ID NO: 64;
a primer set specific for a 16S rRNA gene of nontuberculous mycobacteria, composed of a forward primer comprising a primer having the nucleotide sequence of SEQ ID NO: 65, a primer having the nucleotide sequence of SEQ ID NO: 66, and a primer having the nucleotide sequence of SEQ ID NO: 67, and a reverse primer having the nucleotide sequence of SEQ ID NO: 36; and
a probe for detecting the 16S rRNA gene of nontuberculous mycobacteria, having a nucleotide sequence of SEQ ID NO: 39 or 68.

30. The kit of claim 29, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

31. A method for detecting Mycobacterium tuberculosis and nontuberculous mycobacteria, comprising:
isolating DNA from a test subject; amplifying the DNA by duplex real-time PCR using a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 20, a probe for detecting the IS6110 gene of Mycobacterium tuberculosis, having a nucleotide sequence of SEQ ID NO: 21 or SEQ ID NO: 64, a primer set specific for a 16S rRNA gene of nontuberculous mycobacteria, composed of a forward primer comprising a primer having a nucleotide sequence of SEQ ID NO: 65, a primer having a nucleotide sequence of SEQ ID NO: 66, and a primer having a nucleotide sequence of SEQ ID NO: 67, and a reverse primer having a nucleotide sequence of SEQ ID NO: 36, and a probe for detecting the 16S rRNA gene of nontuberculous mycobacteria, having a nucleotide sequence of SEQ ID NO: 39 or 68; and
analyzing products of the duplex real-time PCR.

32. A kit for detection of Mycobacterium tuberculosis and nontuberculous mycobacteria, comprising:
a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 59;
a probe for detecting the IS6110 gene of Mycobacterium tuberculosis, having a nucleotide sequence of SEQ ID NO: 60;
a primer set specific for a 16S rRNA gene of nontuberculous mycobacteria, composed of a forward primer having a nucleotide sequence of SEQ ID NO: 24, and a reverse primer having a nucleotide sequence of SEQ ID NO: 75; and
a probe for detecting the 16S rRNA gene of nontuberculous mycobacteria, comprising a probe having a nucleotide sequence of SEQ ID NO: 27 and a probe having a nucleotide sequence of SEQ ID NO: 76.

33. The kit of claim 32, wherein the probe for detecting the IS6110 gene of Mycobacterium tuberculosis is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, and
the probe for detecting the 16S rRNA gene of nontuberculous mycobacteria is labeled at a 5′ end with a fluorescent marker selected from the group consisting of FAM, VIC, TET, JOE, HEX, CY3, CY5, ROX, RED610, TEXAS RED, RED670, and NED, and at a 3′ end with a fluorescence quencher selected from the group consisting of 6-TAMRA, BHQ-1,2,3 and MGBNFQ, said fluorescent markers labeled at the 5′ ends differing from the probe for detecting the IS61110 gene of Mycobacterium tuberculosis to the probe for detecting the 16S rRNA of nontuberculous mycobacteria.

34. A method for detecting Mycobacterium tuberculosis and nontuberculous mycobacteria, comprising:
isolating DNA from a test subject;
amplifying the DNA by duplex real-time PCR using a primer set specific for an IS6110 gene of Mycobacterium tuberculosis, consisting of a forward primer having a nucleotide sequence of SEQ ID NO: 58 and a reverse primer having a nucleotide sequence of SEQ ID NO: 59, a probe for detecting the IS6110 gene of Mycobacterium tuberculosis, having a nucleotide sequence of SEQ ID NO: 60, a primer set specific for the 16S rRNA gene of nontuberculous mycobacteria, composed of a forward primer having a nucleotide sequence of SEQ ID NO: 24, and a reverse primer having a nucleotide sequence of SEQ ID NO: 75, and a probe for detecting the 16S rRNA gene of nontuberculous mycobacteria, comprising a probe having a nucleotide sequence of SEQ ID NO: 27 and a probe having a nucleotide sequence of SEQ ID NO: 76; and
analyzing products of the duplex real-time PCR.


SEQUENCE LISTING (76 sequences)

SEQ ID NO: 1 - DNA, 20 bp, Artificial Sequence
  forward primer for amplification specific fragments of IS6110 gene in Mycobacterium tuberculosis complex
cgaactcaaggagcacatca

SEQ ID NO: 2 - DNA, 20 bp, Artificial Sequence
  reverse primer for amplification specific fragments of IS6110 gene in Mycobacterium tuberculosis complex
agtttggtcatcagccgttc

SEQ ID NO: 3 - DNA, 18 bp, Artificial Sequence
  probe for detection of specific fragments of IS6110 gene in Mycobacterium tuberculosis complex
agtgtggctaaccctgaa

SEQ ID NO: 4 - DNA, 17 bp, Artificial Sequence
  forward primer for amplification specific fragments of 16S rRNA gene in Nontuberculous mycobacteria
ggyrayctgccctgcac

SEQ ID NO: 5 - DNA, 18 bp, Artificial Sequence
  reverse primer(NTM-1) for amplification specific fragments of 16S rRNA gene in Nontuberculous mycobacteria
cccacaccgcaaaagctt

SEQ ID NO: 6 - DNA, 17 bp, Artificial Sequence
  reverse primer(NTM-1) for amplification specific fragments of 16S rRNA gene in Nontuberculous mycobacteria
cccacaccgcaaaagct

SEQ ID NO: 7 - DNA, 18 bp, Artificial Sequence
  reverse primer(NTM-1) for amplification specific fragments of 16S rRNA gene in Nontuberculous mycobacteria
tcccacaccgcaaaagct

SEQ ID NO: 8 - DNA, 18 bp, Artificial Sequence
  reverse primer(NTM-2) for amplification specific fragments of 16S rRNA gene in Nontuberculous mycobacteria
catcccacaccgctaccw

SEQ ID NO: 9 - DNA, 20 bp, Artificial Sequence
  probe for detection of specific fragments of 16S rRNA gene in Nontuberculous mycobacteria
cggtattagacccagtttcc

SEQ ID NO: 10 - DNA, 18 bp, Artificial Sequence
  reverse primer(NTM-2) for amplification specific fragments of 16S rRNA gene in Nontuberculous mycobacteria
catcccacaccgctacct

SEQ ID NO: 11 - DNA, 18 bp, Artificial Sequence
  reverse primer(NTM-2) for amplification specific fragments of 16S rRNA gene in Nontuberculous mycobacteria
catcccacaccgctacca

SEQ ID NO: 12 - DNA, 17 bp, Artificial Sequence
  forward primer for amplification specific fragments of 16S rRNA gene in Nontuberculous mycobacteria
ggtaatctgccctgcac

SEQ ID NO: 13 - DNA, 17 bp, Artificial Sequence
  forward primer for amplification specific fragments of 16S rRNA gene in Nontuberculous mycobacteria
ggtaacctgccctgcac

SEQ ID NO: 14 - DNA, 17 bp, Artificial Sequence
  forward primer for amplification specific fragments of 16S rRNA gene in Nontuberculous mycobacteria
ggcaatctgccctgcac

SEQ ID NO: 15 - DNA, 17 bp, Artificial Sequence
  forward primer for amplification specific fragments of 16S rRNA gene in Nontuberculous mycobacteria
ggcaacctgccctgcac

SEQ ID NO: 16 - DNA, 17 bp, Artificial Sequence
  forward primer for amplification specific fragments of 16S rRNA gene in Nontuberculous mycobacteria
ggtgatctgccctgcac

SEQ ID NO: 17 - DNA, 17 bp, Artificial Sequence
  forward primer for amplification specific fragments of 16S rRNA gene in Nontuberculous mycobacteria
ggtgacctgccctgcac

SEQ ID NO: 18 - DNA, 17 bp, Artificial Sequence
  forward primer for amplification specific fragments of 16S rRNA gene in Nontuberculous mycobacteria
ggcgatctgccctgcac

SEQ ID NO: 19 - DNA, 17 bp, Artificial Sequence
  forward primer for amplification specific fragments of 16S rRNA gene in Nontuberculous mycobacteria
ggcgacctgccctgcac

SEQ ID NO: 20 - DNA, 20 bp, Artificial Sequence
  reverse primer for amplification specific fragments of IS6110 gene in Mycobacterium tuberculosis complex
cagggttagccacactttgc

SEQ ID NO: 21 - DNA, 23 bp, Artificial Sequence
  probe for detection of specific fragments of IS6110 gene in Mycobacterium tuberculosis complex
cgccaactacggtgtttacggtg

SEQ ID NO: 22 - DNA, 19 bp, Artificial Sequence
  forward primer for amplification specific fragments of 16S rRNA gene in Nontuberculous mycobacteria
gtggcgaacgggtgagtaa

SEQ ID NO: 23 - DNA, 26 bp, Artificial Sequence
  probe for detection of specific fragments of 16S rRNA gene in Nontuberculous mycobacteria
cggtattagacccagtttcccaggct

SEQ ID NO: 24 - DNA, 20 bp, Artificial Sequence
  forward universal primer for amplification specific fragments of 16S rRNA gene in Mycobacterium
ggataagcytgggaaactgg

SEQ ID NO: 25 - DNA, 20 bp, Artificial Sequence
  reverse universal primer for amplification specific fragments of 16S rRNA gene in Mycobacterium
accccaccaacwagctgata

SEQ ID NO: 26 - DNA, 17 bp, Artificial Sequence
  probe for detection of specific fragments of 16S rRNA gene in Mycobacterium tuberculosis complex
tggtggaaagcgcttta

SEQ ID NO: 27 - DNA, 17 bp, Artificial Sequence
  probe(NTM-1) for detection of specific fragments of 16S rRNA gene in Nontuberculous mycobacteria
tggtggaaagcttttgc

SEQ ID NO: 28 - DNA, 18 bp, Artificial Sequence
  probe(NTM-2) for detection of specific fragments of 16S rRNA gene in Nontuberculous mycobacteria
tggaaagygtttggtagc

SEQ ID NO: 29 - DNA, 20 bp, Artificial Sequence
  forward universal primer for amplification specific fragments of 16S rRNA gene in Mycobacterium
ggataagcctgggaaactgg

SEQ ID NO: 30 - DNA, 20 bp, Artificial Sequence
  forward universal primer for amplification specific fragments of 16S rRNA gene in Mycobacterium
ggataagcttgggaaactgg

SEQ ID NO: 31 - DNA, 20 bp, Artificial Sequence
  reverse universal primer for amplification specific fragments of 16S rRNA gene in Mycobacterium
accccaccaacaagctgata

SEQ ID NO: 32 - DNA, 20 bp, Artificial Sequence
  reverse universal primer for amplification specific fragments of 16S rRNA gene in Mycobacterium
accccaccaactagctgata

SEQ ID NO: 33 - DNA, 18 bp, Artificial Sequence
  probe(NTM-2) for detection of specific fragments of 16S rRNA gene in Nontuberculous mycobacteria
tggaaagcgtttggtagc

SEQ ID NO: 34 - DNA, 18 bp, Artificial Sequence
  probe(NTM-2) for detection of specific fragments of 16S rRNA gene in Nontuberculous mycobacteria
tggaaagtgtttggtagc

SEQ ID NO: 35 - DNA, 22 bp, Artificial Sequence
  forward primer for amplification specific fragments of 16S rRNA gene in Nontuberculous mycobacteria
catgtyttstggkgsaaagctt

SEQ ID NO: 36 - DNA, 19 bp, Artificial Sequence
  reverse primer for amplification specific fragments of 16S rRNA gene in Nontuberculous mycobacteria
cgtaggagtctgggccgta

SEQ ID NO: 37 - DNA, 19 bp, Artificial Sequence
  probe for detection of specific fragments of 16S rRNA gene in Nontuberculous mycobacteria
tagccggcctgagagggtg

SEQ ID NO: 38 - DNA, 19 bp, Artificial Sequence
  probe for detection of specific fragments of 16S rRNA gene in Nontuberculous mycobacteria
cctgagagggtgwccggcc

SEQ ID NO: 39 - DNA, 19 bp, Artificial Sequence
  probe for detection of specific fragments of 16S rRNA gene in Nontuberculous mycobacteria
cgggtagccggcctgagag

SEQ ID NO: 40 - DNA, 22 bp, Artificial Sequence
  forward primer for amplification specific fragments of 16S rRNA gene in Nontuberculous mycobacteria
catgtcttgtgggggaaagctt

SEQ ID NO: 41 - DNA, 22 bp, Artificial Sequence
  forward primer for amplification specific fragments of 16S rRNA gene in Nontuberculous mycobacteria
catgttttgtgggggaaagctt

SEQ ID NO: 42 - DNA, 22 bp, Artificial Sequence
  forward primer for amplification specific fragments of 16S rRNA gene in Nontuberculous mycobacteria
catgtcttctgggggaaagctt

SEQ ID NO: 43 - DNA, 22 bp, Artificial Sequence
  forward primer for amplification specific fragments of 16S rRNA gene in Nontuberculous mycobacteria
catgtcttgtggtggaaagctt

SEQ ID NO: 44 - DNA, 22 bp, Artificial Sequence
  forward primer for amplification specific fragments of 16S rRNA gene in Nontuberculous mycobacteria
catgtcttgtggggcaaagctt

SEQ ID NO: 45 - DNA, 22 bp, Artificial Sequence
  forward primer for amplification specific fragments of 16S rRNA gene in Nontuberculous mycobacteria
catgttttctgggggaaagctt

SEQ ID NO: 46 - DNA, 22 bp, Artificial Sequence
  forward primer for amplification specific fragments of 16S rRNA gene in Nontuberculous mycobacteria
catgtcttctggtggaaagctt

SEQ ID NO: 47 - DNA, 22 bp, Artificial Sequence
  forward primer for amplification specific fragments of 16S rRNA gene in Nontuberculous mycobacteria
catgtcttgtggtgcaaagctt

SEQ ID NO: 48 - DNA, 22 bp, Artificial Sequence
  forward primer for amplification specific fragments of 16S rRNA gene in Nontuberculous mycobacteria
catgttttgtggggcaaagctt

SEQ ID NO: 49 - DNA, 22 bp, Artificial Sequence
  forward primer for amplification specific fragments of 16S rRNA gene in Nontuberculous mycobacteria
catgtcttctggggcaaagctt

SEQ ID NO: 50 - DNA, 22 bp, Artificial Sequence
  forward primer for amplification specific fragments of 16S rRNA gene in Nontuberculous mycobacteria
catgttttgtggtggaaagctt

SEQ ID NO: 51 - DNA, 22 bp, Artificial Sequence
  forward primer for amplification specific fragments of 16S rRNA gene in Nontuberculous mycobacteria
catgttttctggtggaaagctt

SEQ ID NO: 52 - DNA, 22 bp, Artificial Sequence
  forward primer for amplification specific fragments of 16S rRNA gene in Nontuberculous mycobacteria
catgttttctggggcaaagctt

SEQ ID NO: 53 - DNA, 22 bp, Artificial Sequence
  forward primer for amplification specific fragments of 16S rRNA gene in Nontuberculous mycobacteria
catgttttgtggtgcaaagctt

SEQ ID NO: 54 - DNA, 22 bp, Artificial Sequence
  forward primer for amplification specific fragments of 16S rRNA gene in Nontuberculous mycobacteria
catgtcttctggtgcaaagctt

SEQ ID NO: 55 - DNA, 22 bp, Artificial Sequence
  forward primer for amplification specific fragments of 16S rRNA gene in Nontuberculous mycobacteria
catgttttctggtgcaaagctt

SEQ ID NO: 56 - DNA, 19 bp, Artificial Sequence
  probe for detection of specific fragments of 16S rRNA gene in Nontuberculous mycobacteria
cctgagagggtgaccggcc

SEQ ID NO: 57 - DNA, 19 bp, Artificial Sequence
  probe for detection of specific fragments of 16S rRNA gene in Nontuberculous mycobacteria
cctgagagggtgtccggcc

SEQ ID NO: 58 - DNA, 21 bp, Artificial Sequence
  forward primer for amplification specific fragments of IS6110 gene in Mycobacterium tuberculosis complex
cgaactcaaggagcacatcag

SEQ ID NO: 59 - DNA, 21 bp, Artificial Sequence
  reverse primer for amplification specific fragments of IS6110 gene in Mycobacterium tuberculosis complex
gagtttggtcatcagccgttc

SEQ ID NO: 60 - DNA, 19 bp, Artificial Sequence
  probe for detection of specific fragments of IS6110 gene in Mycobacterium tuberculosis complex
agtgtggctaaccctgaac

SEQ ID NO: 61 - DNA, 18 bp, Artificial Sequence
  forward primer for amplification specific fragments of 16S rRNA gene in Nontuberculous mycobacteria
tggcgaacgggtgagtaa

SEQ ID NO: 62 - DNA, 24 bp, Artificial Sequence
  probe for detection of specific fragments of 16S rRNA gene in Nontuberculous mycobacteria
cggtattagacccagtttcccagg

SEQ ID NO: 63 - DNA, 20 bp, Artificial Sequence
  probe for detection of specific fragments of 16S rRNA gene in Nontuberculous mycobacteria
tgggaaactgggtctaatac

SEQ ID NO: 64 - DNA, 18 bp, Artificial Sequence
  probe for detection of specific fragments of IS6110 gene in Mycobacterium tuberculosis complex
ctacggtgtttacggtgc

SEQ ID NO: 65 - DNA, 19 bp, Artificial Sequence
  forward primer(NTM-1) for amplification specific fragments of 16S rRNA gene in Nontuberculous mycobacteria
tktggtggaaagcttttgc

SEQ ID NO: 66 - DNA, 19 bp, Artificial Sequence
  forward primer(NTM-2) for amplification specific fragments of 16S rRNA gene in Nontuberculous mycobacteria
ggtgwgtggtgcaaagctt

SEQ ID NO: 67 - DNA, 18 bp, Artificial Sequence
  forward primer(NTM-3) for amplification specific fragments of 16S rRNA gene in Nontuberculous mycobacteria
tggtggaaagcgtttggt

SEQ ID NO: 68 - DNA, 16 bp, Artificial Sequence
  probe for detection of specific fragments of 16S rRNA gene in Nontuberculous mycobacteria
cctgagagggtgwccg

SEQ ID NO: 69 - DNA, 19 bp, Artificial Sequence
  forward primer(NTM-1) for amplification specific fragments of 16S rRNA gene in Nontuberculous mycobacteria
tgtggtggaaagcttttgc

SEQ ID NO: 70 - DNA, 19 bp, Artificial Sequence
  forward primer(NTM-1) for amplification specific fragments of 16S rRNA gene in Nontuberculous mycobacteria
tttggtggaaagcttttgc

SEQ ID NO: 71 - DNA, 19 bp, Artificial Sequence
  forward primer(NTM-2) for amplification specific fragments of 16S rRNA gene in Nontuberculous mycobacteria
ggtgagtggtgcaaagctt

SEQ ID NO: 72 - DNA, 19 bp, Artificial Sequence
  forward primer(NTM-2) for amplification specific fragments of 16S rRNA gene in Nontuberculous mycobacteria
ggtgtgtggtgcaaagctt

SEQ ID NO: 73 - DNA, 16 bp, Artificial Sequence
  probe for detection of specific fragments of 16S rRNA gene in Nontuberculous mycobacteria
cctgagagggtgaccg

SEQ ID NO: 74 - DNA, 16 bp, Artificial Sequence
  probe for detection of specific fragments of 16S rRNA gene in Nontuberculous mycobacteria
cctgagagggtgtccg

SEQ ID NO: 75 - DNA, 19 bp, Artificial Sequence
  reverse universal primer for amplification specific fragments of 16S rRNA gene in Mycobacterium
cgtaggagtctgggccgta

SEQ ID NO: 76 - DNA, 17 bp, Artificial Sequence
  probe(NTM-2) for detection of specific fragments of 16S rRNA gene in Nontuberculous mycobacteria
ccacaccgctaccaaac